(19) United States
(12) Patent Application Publication
(10) Pub. No.: US 2023/0090337 A1
(43) Pub. Date: Mar. 23, 2023

(54) PHARMACEUTICAL FORMULATIONS COMPRISING 6-CHLORO-7-(4-(4-CHLOROBENZYL)PIPERAZIN-1 -YL)-2-(1,3-DIMETHYL-1 HPYRAZOL-4-YL)-3H- IMIDAZO[4,5-B]PYRIDINE

(71) Applicants: ELLIPSES PHARMA LTD, London (GB); INSTITUTE OF CANCER RESEARCH: ROYAL CANCER HOSPITAL (THE), London (GB)

(72) Inventors: Michaela KREINER, Glasgow (GB); Gavin HALBERT, Glasgow (GB); Shanoo BUDHDEO, Cheshire (GB); Paul DICKINSON, Cheshire (GB)

(21) Appl. No.: 17/909,279

(22) PCT Filed: Mar. 3, 2021
(86) PCT No.: PCT/GB2021/050529

§ 371 Date: Sep. 2, 2022

(30) Foreign Application Priority Data
Mar. 4, 2020 (GB) 2003108.4

(51) Int. Cl. A61K 31/437; A61K 9/48; A61K 9/20; A61K 47/55; A61K 47/14; A61K 47/26; A61K 47/10

(52) U.S. Cl. CPC A61K 31/437; A61K 9/48; A61K 9/20; A61K 47/551; A61K 47/14; A61K 47/26; A61K 47/10

(57) ABSTRACT

The present invention relates to formulations comprising a compound of Formula (1) (6-chloro-7-(4-(4-chlorobenzyl)piperazin-1-yl)-2-(1,3-dimethyl-1H-pyrazol-4-yl)-3H-imidazo[4,5-b]pyridine) which is an inhibitor of Aurora kinase enzyme activity and FMS-like tyrosine kinase 3 (FLT3) activity, and a non-ionic surfactant. The present invention also relates to processes for the preparation of the formulations of the compound, and to their use in the treatment of proliferative disorders, such as cancer, as well as other diseases or conditions in which Aurora kinase and/or FLT3 activity is implicated.

The present invention relates to formulations of a pharmaceutically active compound. More specifically, the present invention relates to formulations of a compound that is an inhibitor of Aurora kinase enzyme activity. The compound is also an inhibitor of FMS-like tyrosine kinase 3 (FLT3) activity. The present invention also relates to processes for the preparation of the formulations of the compound, and to their use in the treatment of proliferative disorders, such as cancer, as well as other diseases or conditions in which Aurora kinase and/or FLT3 activity is implicated.

### BACKGROUND

Proliferative diseases, such as cancer, are characterised by uncontrolled and unregulated cellular proliferation. Precisely what causes a cell to proliferate in an uncontrolled and unregulated manner has been the focus of intense research over recent decades.

Aurora kinases, a family of three serine-threonine kinases designated as A, B, and C, play key and distinct roles in different stages of mitosis. At the early stages of mitosis, Aurora-A forms a complex with the targeting protein for Xklp2 (TPX2) that regulates centrosome maturation and mitotic spindle assembly. Aurora-B forms complexes with the inner centromere protein (INCENP), survivin and borealin thereby regulating chromosome condensation, chromosome alignment, mitotic checkpoint and cytokinesis. Over expression of Aurora-A and Aurora-B has been reported in a wide range of human malignancies including breast, colorectal, ovarian, glioma, thyroid carcinoma, and seminoma. The function of Aurora-C during mitosis is less well understood. However, high expression of Aurora-C has been reported in the testis.

FLT3 is a trans-membrane kinase that belongs to the class III receptor tyrosine kinase (RTK) family. Binding of FLT3-ligand (FL) to its receptor leads to dimerisation, autophosphorylation and subsequent activation of downstream signalling pathways. High levels of FLT3 expression have been found in acute myeloid leukaemia (AML) blasts, and two major classes of mutations, i.e. internal-tandem duplications (ITDs) and tyrosine kinase domain (TKD) point mutations, have been identified in AML patients. Internal-tandem duplications are detected in 20-25% of AML patients, and tyrosine kinase domain point mutations in 5-10% of AML patients.

There is, therefore, a further need for compounds that have a dual function of inhibiting both Aurora kinases and FLT3. Such compounds would be useful for the treatment of diseases and/or conditions in which Aurora and/or FLT3 are implicated, such as, for example, AML.

CCT241736 is one of such dual inhibitory compounds, having high activity against Aurora A, B, and C, and FLT3 kinases, as well an advantageous therapeutic window resulting from minimal interaction with cytochrome P450 activity and hERG. The preparation and biological testing of CCT241736 is described in WO 2013/190319. Other promising dual aurora/FLT3 kinase inhibitors are also described in WO 2013/190319.

In order to design a dosage form that is suitable for scaling up and for further development, the specific chemical and physical properties of the active pharmaceutical ingredient (API) must be taken into account. Each API will have a unique set of chemical and physical properties that will generate a specific set of challenges for pharmaceutical formulation. In other words, these formulation challenges will be unique and specific to the API. Key issues that arise in development of suitable formulations include physical and chemical stability, and aqueous solubility.

### SUMMARY OF INVENTION

The invention provides a pharmaceutical composition, comprising:
a) a compound according to Formula (1):

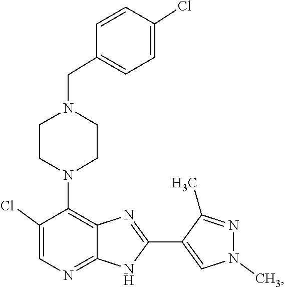 [SMILES: Cc1nn(C)cc1-c1nc2c(N3CCN(Cc4ccc(Cl)cc4)CC3)c(Cl)cnc2[nH]1]

or a pharmaceutically acceptable salt, and/or solvate thereof; and
b) a non-ionic surfactant.

The claimed formulations unexpectedly have the ability to solubilise and stabilise compounds of Formula (1) through the use of non-ionic surfactants. The provision of physically and/or chemically stable formulations is crucial to the development of a viable drug product. In addition, the inventors have also found that particularly preferred formulations of the invention containing a particular type of non-ionic surfactant (vitamin E derivatives such as d-α tocopherol polyethylene glycol 1000 succinate) are able to increase the aqueous solubility of the compound of Formula (1), for example increase the solubility of the compound of Formula (1) in simulated intestinal fluid compared to other formulations. Increasing solubility is a key goal in development of drug products on account of the associated increases in absorption and bioavailability of the drug in vivo.

The composition of the invention can be for use in therapy. For example, the pharmaceutical composition of the invention is for use in the treatment of diseases or conditions in which Aurora kinase and/or FLT3 activity is implicated.

For example, the present invention provides the use of a compound of Formula (1), in the manufacture of a medicament comprising a pharmaceutical composition as defined herein for the treatment of diseases or conditions in which Aurora kinase and/or FLT3 activity is implicated.

The present invention also provides a method of treating a disease or condition in which Aurora kinase and/or FLT3 activity is implicated, said method comprising administering to a subject in need of such treatment a therapeutically effective amount of a pharmaceutical composition as defined herein.

The present invention also provides a pharmaceutical composition as defined herein for use in the treatment of a proliferative disorder, such as cancer. In a particular embodiment, the cancer is a human cancer.

The present invention also provides the use of a compound of Formula (1), in the manufacture of a medicament comprising a pharmaceutical composition as defined herein for the treatment of a proliferative disorder, such as cancer. In a particular embodiment, the cancer is a human cancer.

The present invention also provides a method of treating a proliferative disorder, such as cancer, said method comprising administering to a subject in need of such treatment a therapeutically effective amount of a pharmaceutical composition as defined herein. In a particular embodiment, the cancer is a human cancer.

The present invention also provides a pharmaceutical composition as defined herein for use in the production of an Aurora kinase and/or FLT3 inhibitory effect.

The present invention also provides the use of a compound of Formula (1), in the manufacture of a medicament comprising a pharmaceutical composition as defined herein for the production of an Aurora kinase and/or FLT3 inhibitory effect.

The present invention also provides a method of producing an in vitro or in vivo Aurora kinase and/or FLT3 inhibitory effect, said method comprising administering an effective amount of a pharmaceutical composition as defined herein.

The present invention also provides a method of inhibiting cell proliferation in vitro or in vivo, said method comprising contacting a cell with an effective amount of a pharmaceutical composition as defined herein.

The present invention also provides methods of formulating the pharmaceutical compositions described herein. The present invention also provides the use of a compound of Formula (1) in the manufacture of a medicament comprising a composition as defined herein. The invention may be prepared by mixing, dissolving, dispersing, suspending, spray drying, melting, tabletting, compacting, a compound of Formula (1) with a non-ionic surfactant and optionally any other additional pharmaceutically acceptable carriers, diluents and excipients.

### Definitions

The chemical name of the compound of Formula (1) is 6-chloro-7-(4-(4-chlorobenzyl)piperazin-1-yl)-2-(1,3-dimethyl-1H-pyrazol-4-yl)-3H-imidazo[4,5-b]pyridine. The compound has CAS No. 1402709-93-6. For the avoidance of doubt, unless stated to the contrary, reference to the compound of Formula (1), or the “compound” is a reference to this compound having the structure above, optionally in the form of a pharmaceutically acceptable salt and/or solvate.

As used herein, the term “pharmaceutically acceptable salts” refers to salts that retain the biological effectiveness and properties of the compounds of this invention and, which typically are not biologically or otherwise undesirable. In many cases, the compounds of Formula (I) are capable of forming acid and/or base salts by virtue of the presence of amino and/or carboxyl groups or groups similar thereto.

Pharmaceutically acceptable acid addition salts can be formed with inorganic acids, such as hydrochloric acid, hydrobromic acid, sulfuric acid, nitric acid, phosphoric acid, and the like. Pharmaceutically acceptable acid addition salts can be formed with organic acids, e.g., acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Preferably, the pharmaceutically acceptable salt of compound (1) is a fumarate salt, for example a stoichiometric fumarate or a hemi fumarate, preferably a stoichiometric fumarate (1:1 molar ratio of the compound of Formula (1) and fumaric acid).

The pharmaceutically acceptable salts of the present invention can be synthesized from a parent compound, a basic or acidic moiety, by conventional chemical methods. Generally, such salts can be prepared by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in “Remington's Pharmaceutical Sciences”, 20th ed., 30 Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The terms “effective amount” or “therapeutically effective amount,” as used herein, refer to a sufficient amount of an agent being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms.

The term “therapeutically effective amount” includes, for example, a prophylactically effective amount. The effective amount will be selected based on the particular patient and the disease level. It is understood that “an effect amount” or “a therapeutically effective amount” varies from subject to subject, due to variation in metabolism of drug, age, weight, general condition of the subject, the condition being treated, the severity of the condition being treated, and the judgment of the prescribing physician. In one embodiment, an appropriate “effective” amount in any individual case is determined using techniques, such as a dose escalation study.

The compound of Formula (1) can exist in both unsolvated and solvated forms. The term “solvate” is used herein to describe a molecular complex comprising an API such as the compound of Formula (1) and an amount of one or more pharmaceutically acceptable solvents. Where the solvent is water the term “hydrate” is used.

### DESCRIPTION OF FIGURES

FIG. 1: individual and mean pH shift dissolution profiles for poloxamer 124 based 100 mg capsules of the compound of formula (1).

FIG. 2: individual and mean pH shift dissolution profiles for TPGS based 100 mg capsules of the compound of formula (1).

FIG. 3: overlay of mean (shading=s.d) pH shift dissolution profiles for poloxamer P124 and TPGS based capsules containing 100 mg of the compound of formula (1).

FIG. 4: individual and mean FaSSIF dissolution profiles for poloxamer based 100 mg capsules of the compound of formula (1).

FIG. 5: individual and mean FaSSIF dissolution profiles for TPGS based 100 mg capsules of the compound of formula (1).

FIG. 6: overlay of mean (shading=s.d) FaSSIF dissolution profiles for poloxamer and TPGS based 100 mg capsules of the compound of Formula (1).

### DETAILED DESCRIPTION

Compound of Formula (1)

The invention relates to formulations comprising, consisting essentially of, or consisting of a compound of Formula (1) or a pharmaceutically acceptable salt thereof, and a non-ionic surfactant. A compound of Formula (1) has the structure:

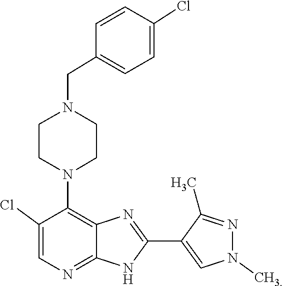 [SMILES: Cc1nn(C)cc1-c1nc2c(N3CCN(Cc4ccc(Cl)cc4)CC3)c(Cl)cnc2[nH]1]

The compound of formula 1 has the chemical name: 6-chloro-7-(4-(4-chlorobenzyl)piperazin-1-yl)-2-(1,3-dimethyl-1H-pyrazol-4-yl)-3H-imidazo[4,5-b]pyridine. The compound has CAS No. 1402709-93-6. For the avoidance of doubt, unless stated to the contrary, reference to the compound of Formula (1), or the “compound” is a reference a compound having the structure above, optionally in the form of a pharmaceutically acceptable salt and/or solvate.

The compositions of the invention may comprise the compound of Formula (1) as a free base. Alternatively, various salts of the compound are available (for example hydrochloride, mesylate, fumarate, etc.). Preferably, the pharmaceutically acceptable salt of compound (1) is a fumarate salt. The fumarate salt has been shown to have higher aqueous solubility than the free base and greater stability, e.g. due to lower hygroscopicity, than other pharmaceutically acceptable salts.

The compositions of the invention may comprise any therapeutically effective amount of a compound of Formula 1. These therapeutically effective amounts shown below are based on the weight of the free base of the compound unless indicated otherwise. In any embodiment below, the disclosed weight of free base may be replaced with the weight of the fumarate salt based on the fact that 1.2543 grams of the fumarate salt of the compound of Formula 1 contains 1 gram of its free base. The use of the word “about” in the embodiments below reflects the natural variation in dosing that arises from batch to batch in pharmaceutical manufacturing. In any embodiment below, the word “about” may be removed, although the skilled person would appreciate that the natural variation in dosing remains.

The composition may comprise from about 1 mg to about 2 g of the compound of Formula 1. The composition may comprise from about 1 mg to: about 1 g; about 500 mg; about 400 mg; about 300 mg; about 200 mg; about 100 mg; about 90 mg; about 80 mg; about 70 mg; about 60 mg; about 50 mg; about 40 mg; about 30 mg; about 20 mg; about 10 mg; about 5 mg; about 4 mg; about 3 mg; or about 2 mg of the compound of Formula 1. The composition may comprise from: about 2 mg; about 3 mg; about 4 mg; about 5 mg; about 10 mg; about 20 mg; about 30 mg; about 40 mg; about 50 mg; about 60 mg; about 70 mg; about 80 mg; about 90 mg; about 100 mg; about 200 mg; about 300 mg; about 400 mg; about 500 mg to about 2 g; or about 1 g to about 2 g of the compound of Formula 1.

The composition may comprise from about 1 mg to about 50 mg of the compound of Formula 1. The composition may comprise from about 5 mg to about 45 mg of the compound of Formula 1. The composition may comprise from about 10 mg to about 35 mg of the compound of Formula 1. The composition may comprise from about 10 mg to about 30 mg of the compound of Formula 1. The composition may comprise from about 15 mg to about 25 mg of the compound of Formula 1. Preferably, the composition may comprise about 20 mg of the compound of Formula 1.

The composition may comprise from about 25 mg to about 75 mg of the compound of Formula 1. The composition may comprise from about 30 mg to about 70 mg of the compound of Formula 1. The composition may comprise from about 35 mg to about 65 mg of the compound of Formula 1. The composition may comprise from about 40 mg to about 60 mg of the compound of Formula 1. The composition may comprise from about 45 mg to about 55 mg of the compound of Formula 1. Preferably, the composition may comprise about 50 mg of the compound of Formula 1.

The composition may comprise from about 50 mg to about 150 mg of the compound of Formula 1. The composition may comprise from about 60 mg to about 140 mg of the compound of Formula 1. The composition may comprise from about 70 mg to about 130 mg of the compound of Formula 1. The composition may comprise from about 80 mg to about 120 mg of the compound of Formula 1. The composition may comprise from about 90 mg to about 110 mg of the compound of Formula 1. Preferably, the composition may comprise about 100 mg of the compound of Formula 1.

The amount of the compound can also be expressed as a weight percentage of the total composition. The composition of the invention may comprise from about 1% to about 50% of a compound of Formula (1) as compared to the total weight of the composition.

The composition may comprise from about 1% to: about 2%; about 3%; about 4%; about 5%; about 10%; about 15%; about 20%; about 25%; about 30%; about 35%; about 40%; about 45%, or from about 1% to about 50% by weight of the compound of Formula (1). The composition may comprise from: about 1%; about 2%; about 3%; about 4%; about 5%; about 10%; about 15%; about 20%; about 25%; about 30%; about 35%; about 40%; or about 45%; to about 50% by weight of the compound of Formula (1).

The composition may comprise from about 1% to about 10% by weight of the compound of Formula (1). The composition may comprise from about 1% to about 9% by weight of the compound of Formula (1). The composition may comprise from about 2% to about 8% by weight of the compound of Formula (1). The composition may comprise from about 3% to about 7% by weight of the compound of Formula (1). The composition may comprise from about 4% to about 6% by weight of the compound of Formula (1). The composition may comprise about 5% by weight of the compound of Formula (1), for example containing 20 mg of the compound of Formula (1).

The composition may comprise from about 5% to about 15% by weight of the compound of Formula (1). The composition may comprise from about 6% to about 14% by weight of the compound of Formula (1). The composition may comprise from about 7% to about 13% by weight of the compound of Formula (1). The composition may comprise from about 8% to about 12% by weight of the compound of Formula (1). The composition may comprise from about 9% to about 11% by weight of the compound of Formula (1). The composition may comprise about 10% by weight of the compound of Formula (1), for example containing 50 mg of the compound of Formula (1).

The composition may comprise from about 10% to about 20% by weight of the compound of Formula (1). The composition may comprise from about 11% to about 19% by weight of the compound of Formula (1). The composition may comprise from about 12% to about 18% by weight of the compound of Formula (1). The composition may comprise from about 13% to about 17% by weight of the compound of Formula (1). The composition may comprise from about 14% to about 16% by weight of the compound of Formula (1). The composition may comprise about 15% by weight of the compound of Formula (1), for example containing 50 mg of the compound of Formula (1).

The compound of Formula (1) may be milled prior to its incorporation into the pharmaceutical compositions of the invention. For example, the compound of Formula (1) may be wet ball milled, or dry ball milled, preferably wet ball milled, or by jet milled. Alternatively, the compound of Formula (1) is not milled.

The compositions of the invention are preferably for oral administration. Compositions for oral administration include hard capsules, soft capsules, tablets, pills, and oral liquids. For example, the compositions of the invention may be in the form of hard capsules or soft capsules. Hard capsules are typically produced from two halves that are sealed together after the compound of Formula (1), the non-ionic surfactant, and any other additional excipients are added. Hard capsules can be filled manually (e.g. with a pipette, or automatically (e.g. with an automated filling machine such as a Capsugel CFS 1000). Hard capsules may be made from gelatin, or HPMC (hydroxypropylmethyl cellulose), with the latter being preferred due to a reduction in capsule leakage compared to gelatin capsules.

The compositions of the invention may take any pharmaceutically acceptable physical form at ambient temperature. For example, the composition may be a mixture, a liquid-liquid dispersion, a solid-liquid dispersion (e.g. a suspension), a solid-solid dispersion, a semi-solid matrix, or a solution. When vitamin E derivatives such as TPGS are used as the non-ionic surfactant, the formulation may take the form of a semi-solid matrix.

Non-Ionic Surfactants

A non-ionic surfactant is an amphiphilic molecule having a hydrophobic portion and a hydrophilic portion connected by one or more linkers, which allows it to be a surface active molecule, that is substantially non-ionised (i.e. uncharged) in water in neutral pH. Non-ionic surfactants can be represented by the general Formula (2):

### Hydrophilic portion-linker(s)-hydrophobic portion

The hydrophobic portion typically contains one or more optionally substituted, linear or branched, saturated or unsaturated 3-30 carbon chain.

The hydrophilic portion typically contains one or more moieties comprising non-ionisable oxygen-containing groups such as (poly)alcohols and/or (poly)ethers.

The linker or linkers typically contain one or more ester and/or ether bonds. The linker may be a separate chemical moiety or may derive from the connection of a hydrophilic portion and a hydrophobic portion.

Non-ionic surfactants can be selected from the group consisting of vitamin E derivatives, fatty acid esters, fatty alcohol ethers, glycerol derivatives, sorbitan derivatives, co-polymers, and combinations thereof.

In one embodiment, the non-ionic surfactant is characterised by its hydrophilic-lipophilic balance (HLB). HLB is a metric of the relative hydrophilicity and hydrophobicity of non-ionic amphiphilic surfactants—lower HLB values are more hydrophobic, whereas higher HLB values are more hydrophilic. HLB can be calculated by the Griffin method, i.e. 20×(Mₕ/M), where Mₕ is the molecular weight of the hydrophilic groups and M is the molecular weight of the whole molecule (Griffin, W. C., J. Soc. Cosmet. Chem., 1 (1949) 311-326), which is incorporated herein by reference). HLB values for mixtures of emulsifiers can be calculated by methods known in the art. The inventors have discovered that non-ionic surfactant with a hydrophilic-lipophilic balance (HLB) of greater than about 5, or greater than about 10, for example from about 10 to about 20 are particularly useful for preparing formulations with a compound of Formula (1). The non-ionic surfactant may have a hydrophilic-lipophilic balance (HLB) of greater than about 12 for example from about 12 to about 18. The non-ionic surfactant may have a hydrophilic-lipophilic balance (HLB) of greater than about 14, for example from about 12 to 16.

The non-ionic surfactant preferably makes up from about 5% to about 95% by weight of the composition, more preferably makes up from about 20% to about 90% by weight of the composition, more preferably makes up from about 40% to about 90% by weight of the composition, more preferably makes up from about 60% to about 90% by weight of the composition, and most preferably makes up from about 80 to about 90% by weight of the composition.

Thus, the ratio of the weight of non-ionic surfactant to the weight of the compound of Formula (1) is from about 1:1 to about 25:1. The ratio may be from about 2:1 to about 25:1. The ratio may be from about 5:1 to about 20:1. The ratio may be from about 7:1 to about 20:1. The ratio may be from about 1:1 to about 10:1. The ratio may be from about 1:1 to about 8:1. The ratio may be from about 2:1 to about 7:1. The ratio may be 24:1. The ratio may be 7:1. This ratio is preferably calculated with the weight of the free base of the compound of Formula 1.

Hydrophilic Portions

Non-ionic surfactants have a hydrophilic portion that allows them to interact with water and other polar solvents. The hydrophilic portion contains one or more moieties comprising non-ionisable oxygen-containing groups such as alcohols and/or ethers. Examples of alcohols used as non-ionisable oxygen containing groups include monohydric alcohols, dihydric alcohols (diols) such as alkylene glycols (e.g. propane diols such as propylene glycol), trihydric alcohols (triols) such as glycerol, and polyhydric alcohols (polyols) such as sugar alcohols, such as erythritol, sorbitan, and/or mannitol, and saccharides such as fructose, glucose and/or sucrose. Examples of ethers include polymers of alkylene glycol, for example polyethylene glycol (i.e. macrogol, polyoxyethylene, polyethylene oxide, etc.), or polypropylene glycol. In many cases, the hydrophilic portion is a combination of two or more of these different types of moiety, for example any of the alcohols listed above is commonly combined with polyethylene glycol.

Ethers

Non-ionic surfactants in the composition of the invention preferably have a hydrophilic portion containing an ether which is polyethylene glycol. Polyethylene glycol consists of a series of oxyethylene groups connected as shown in Formula (3) below to form a polyethylene glycol chain:

—O—[CH₂CH₂—O]ₙ—

The length, n, of the polyethylene glycol can be varied to fine tune the surfactant properties of the surfactant. The length can be described by the average number of molar equivalents of oxyethylene units (corresponding to n above), or by the number average molecular weight of the polyoxyethylene portion. As used herein, the molecular weight of a polyalkoxy group is the molecular weight of the polyalkoxy chain and its substituents (but does not include the weight of adjacent moieties that are not part of the polyalkoxy chain). The polyalkoxy group is preferably unsubstituted. The number average molecular weight can be determined readily by the skilled person through gel permeation chromatography/size exclusion chromatography, e.g. according to the OECD GUIDELINE FOR TESTING OF CHEMICALS: Test no. 118: Determination of the Number-Average Molecular Weight and the Molecular Weight Distribution of Polymers using Gel Permeation Chromatography. Alternatively, polyethylene glycols having the molecular weights described below are commercially available.

The polyoxyethylene portion may contain a number average of from about 1 to about 180 oxyethylene groups (n=1-180), corresponding to an approximate average molecular weight of from about 50 to about 10000 g/mol. For example, the polyoxyethylene portion may contain a number average of about 2, 4, 6, 8, 9, 10, 12, 15, 20, 32, 36, 40, 60, 75, 150, or 180, preferably from about 2 to about 75, from about 4 to about 40, more preferably from about 6 to about 32, more preferably from about 8 to about 20 molar equivalents of oxyethylene groups. The polyoxyethylene portion may have a number average molecular weight of about 100, 200, 300, 400, 450, 500, 600, 1000, 1540, 1800, 2000, 3000, 4000, 6000, 8000 g/mol, preferably from about 100 to about 4000, more preferably from about 200 to about 4000, more preferably from about 300 to about 1540, more preferably from about 400 to about 1000 g/mol. In some embodiments, the relation between the average number of oxyethylene units and the approximate number average molecular weight is as follows, with n in brackets: 100 (2); 200 (4), 300 (6); 400 (8); 450 (9); 500 (10); 600 (12); 1000 (20); 1540 (32); 1800 (36); 2000 (40); 3000 (60); 4000 (75); 6000 (150); 8000 (180).

The hydrophilic portion may contain multiple polyethylene glycol chains, each attached via a linker (e.g. an ether or an ester linker) to the hydrophobic portion. The polyethylene glycol groups can be combined with any other hydrophilic group described herein to form compound hydrophilic portions.

Alcohols

Non-ionic in the composition of the invention may have a hydrophilic portion containing one or more alcohols.

For example, the hydrophilic portion may comprise, consist of, or consist essentially of one or more diols. Diols can form one or two linkages (e.g. each could be an ester or an ether linkage) with the hydrophobic portion. An example of a diol in a hydrophilic portion is propylene glycol or propane 1,3 diol, which can form one or two ether and/or ester linkages with the hydrophobic portion.

The hydrophilic portion may comprise, consist of, or consist essentially of one or more triols. The most commonly used triol is glycerol. Glycerol can form one, two or three ester or ether linkages with the hydrophobic portion (for example making mono, di, or triglycerides).

The hydrophilic portion may comprise, consist of, or consist essentially of one or more sugar alcohols, such as erythritol, sorbitan, and/or mannitol, and saccharides such as fructose, glucose and/or sucrose. These polyols can be attached to the hydrophobic portion by any available hydroxyl group through an ester or ether linkage.

Mixed Hydrophilic Portions

The hydrophilic portion may comprise, consist of, or consist essentially of an alcohol and one or more polyethylene glycol chains, as defined herein. The alcohol can be connected to the one or more polyethylene glycol chains through one or more ether groups. The ester or ether linkage to the hydrophobic portion can be made through one or more polyethylene glycol chains and/or, in the case of a polyhydric alcohol, through one of the alcohol groups not linked to the polyethylene glycol.

Thus, the hydrophilic portion preferably comprises, consists of, or consists essentially of polyoxyethylated (polyalkoxylated, PEGlyated, etc.) monohydric alcohols, dihydric alcohols (diols) such as polyoxyethylated alkyklene glycols (e.g. propane diols such as propylene glycol), polyoxyethylated trihydric alcohols (triols) such as polyoxyethylated glycerol (mono-, di- or tri-glycerides), and polyoxyethylated polyhydric alcohols (polyols) such as sugar alcohols, such as erythritol, sorbitan, and/or mannitol, and saccharides such as fructose, glucose and/or sucrose.

Hydrophobic Portions

Non-ionic surfactants have a hydrophobic portion that allows them to interact with other non-polar materials. The non-ionic surfactants typically have a hydrophobic portion comprising, consisting of, or consisting essentially of, one or more linear or branched 3 to 30 carbon chain. The carbon chain may be optionally substituted with one or more hydroxyl, C₁-C₆ alkylene hydroxyl, C₁-C₆ alkoxyl, and/or C₁-C₆ alkyl groups. The carbon chain may be saturated (no double bonds) or unsaturated (one or more cis or trans double bonds). In some embodiments, the carbon chain can be defined by a “C:D” ratio, where “C” is the total amount of carbon atoms of the fatty acid, and “D” is the number of double bonds in it. Where D>1 it is assumed that any adjacent double bonds are separated by one or more methylene bridge(s). The carbon chain may have C=4 to 30, and D=0 to 10, for example C=8 to 22, D=0 to 6, more preferably C=8 to 20, D=0 to 4, more preferably C=12 to 18, D=0 to 1.

Exemplary carbon chains include (C:D) 3:0, 4:0, 5:0, 6:0, 7:0, 8:0, 9:0, 10:0, 11:0, 12:0, 13:0, 14:0, 15:0, 16:0, 17:0, 18:0, 19:0, 20:0, 21:0, 22:0, 23:0, 24:0, 25:0, 26:0, 27:0, 28:0, 29:0, 30:0, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 4:2, 5:2, 6:2, 7:2, 8:2, 9:2, 10:2, 11:2, 12:2, 13:2, 14:2, 15:2, 16:2, 17:2, 18:2, 19:2, 20:2, 21:2, 22:2, 23:2, 24:2, 25:2, 26:2, 27:2, 28:2, 29:2, 30:2, 6:3, 7:3, 8:3, 9:3, 10:3, 11:3, 12:3, 13:3, 14:3, 15:3, 16:3, 17:3, 18:3, 19:3, 20:3, 21:3, 22:3, 23:3, 24:3, 25:3, 26:3, 27:3, 28:3, 29:3, 30:3, 8:4, 9:4, 10:4, 11:4, 12:4, 13:4, 14:4, 15:4, 16:4, 17:4, 18:4, 19:4, 20:4, 21:4, 22:4, 23:4, 24:4, 25:4, 26:4, 27:4, 28:4, 29:4, 30:4, etc. and isomers and derivatives thereof.

The carbon chain may optionally substituted with one or more hydroxyl, C₁-C₆ alkylene hydroxyl, C₁-C₆ alkoxyl, and/or C₁-C₆ alkyl groups. Preferred carbon chains include 12:0, 16:0, 18:0, 18:1, and 20:1. Preferably, these carbon chains are linear. The carbon chain may be optionally substituted with one or more hydroxyl groups, for example 1 to 6 hydroxyl groups. The carbon chain may be optionally substituted with one or more C₁-C₆ alkylene hydroxyl groups, for example 1 to 6 C₁-C₆ alkylene hydroxyl groups. The carbon chain may be optionally substituted with one or more C₁-C₆ alkoxyl, for example 1 to 6 C₁-C₆ alkoxyl groups. The carbon chain may be optionally substituted with one or more C₁-C₆ alkyl groups for example 1 to 6 C₁-C₆ alkyl groups. The carbon chain may be unsubstituted.

Each hydrophobic C3-C30 carbon chain can be derived from an alcohol or a carboxylic acid, in which case the hydroxyl or carboxyl group, usually a terminal hydroxyl or carboxyl group, can be conceptualised as forming a linker to the hydrophilic portion. In general, carboxylic acids (i.e. fatty carboxylic acids) are preferred in the non-ionic surfactants of the invention.

In one preferred embodiment, the non-ionic surfactant has a hydrophobic portion containing a vitamin E compound. In particular, the non-ionic surfactant has a hydrophobic portion containing one or more tocopherols (e.g. α, β, γ, δ, ε or x tocopherol), one or more tocotrienol (e.g. α, β, γ, δ, ε or x tocotrienol) and/or derivatives thereof. The α tocopherols and derivatives thereof are preferred.

In one embodiment, the hydrophobic portion contains a series of repeating polymer units, for example polyoxyalkylene units such as propylene glycol units. These three-carbon repeating units constitute the linear or branched carbon chain described generally above.

Linkers

The non-ionic surfactant contains a linker that joins the hydrophobic portion to the hydrophilic portion. The linker comprises at least one chemical bond, for example an ester bond or an ether bond. Where the linker consists or consists essentially of a chemical bond, the hydrophobic and hydrophilic portions contain compatible reactive groups that can form this chemical bond. For example, the hydrophobic portion may contain an alcohol and/or a carboxylic acid (or an activated carboxylic acid derivative known in the art such as an anhydride or an acyl chloride) that can react with the hydrophilic portion, which may also contain an alcohol and/or a carboxylic acid (or an activated carboxylic acid derivative known in the art such as an anhydride or an acyl chloride) to form ester and ether linkages.

In some preferred embodiments, the linker comprises, consists of, or consist essentially of a short spacer molecule that connects the hydrophilic and hydrophobic portions. For example, the linker may comprise, consist of, or consist essentially of a C1-C10 diol that can form ester or ether linkages with the hydrophobic and hydrophilic portions (for example ethane diol, propane diol, butane diol, pentane diol, hexane diol, etc.), or the linker may comprise, consist of, or consist essentially of a C1-C10 diacid that can form ester linkages with the hydrophobic and hydrophilic portions (for example ethanedioic acid (oxalic acid), propanedioic acid (malonic acid), butanedioic acid (succinic acid), pentanedioic acid (glutaric acid), hexanedioic acid (adipic acid), etc.). A preferred linker contains a succinic acid spacer.

Non-Ionic Surfactant Classes

The hydrophobic portion, hydrophilic portion, and linker portion defined above lead to several classes of non-ionic surfactant. Thus, the composition of the invention can comprise non-ionic surfactants such as: vitamin E and derivatives thereof, fatty acid esters, fatty alcohol ethers, glycerol derivatives, sorbitan esters, and co-polymers. Any of these non-ionic surfactant classes may be polyoxyethylated.

Vitamin E Derivatives

Compositions of the invention may include a vitamin E derivatives as a non-ionic surfactant. These are typically derived from a hydrophilic portion containing a polyoxythylene chain, a hydrophobic portion containing a tocopherol or tocotrienol, and optionally a short chain dicarboxylic acid linker. The vitamin E derivative may have a HLB greater than about 5, for example greater than about 10, for example from about 10 to about 20, from about 12 to about 18, or from about 12 to 16.

Vitamin E Derivatives—Hydrophobic Portions

The hydrophobic portion of vitamin E derivatives comprises, consists of, or consists essentially of one or more tocopherols or tocotrienols. Natural and/or synthetic forms of vitamin E can be used to make these. Natural vitamin E exists in eight different forms: four tocopherols and four tocotrienols. All natural forms have a chromanol ring, with a hydroxyl group for connection to the linker or to the hydrophilic portion, and a hydrophobic side chain which allows for penetration into biological membranes. There is an α, β, γ, δ ε and x form of both the tocopherols and tocotrienols, determined by the number of methyl groups on the chromanol ring. The α-tocopherols are preferred.

In one aspect vitamin E derivatives for inclusion in the compositions of the invention do not include tocotrienols. In one aspect vitamin E derivatives for inclusion in compositions of the invention are tocopherols (and any of the α, β, γ, δ, ε or x tocopherols can be used) and derivatives thereof. The α tocopherols are preferred, particularly d-α-tocopherol. In one embodiment, however, the vitamin E is not d-α-tocopherol. The tocopherols can exist in different isomeric forms. Pure stereoisomers or mixtures of stereoisomers may be used, for example D-α-tocopherol and DL-α-tocopherol can both be used.

Vitamin E Derivatives—Linkers

The linker portion may be formed with the hydroxyl group of the chromanol ring on the tocopherol or tocotrienol. In one embodiment, the linker is an ether bond. In preferred embodiments, the linker is a short chain (C2-C10) dicarboxylic acid, diol, or a hydroxycarboxylic acid, preferably a C2-C10 dicarboxylic acid.

Preferred diacids for forming linkers are oxalic, malonic, succinic, glutaric, adipic acid etc. to make oxalate, malonate, succinate, glutarate, adipate etc. esters. The carbon chain may be optionally substituted with one or more C₁-C₆ alkoxyl, for example 1 to 6 C₁-C₆ alkoxyl groups. The carbon chain may be optionally substituted with one or more C₁-C₆ alkyl groups for example 1 to 6 C₁-C₆ alkyl groups. The most preferred linker for a vitamin E derivative non-ionic surfactant is an unsubstituted succinic acid linker where the first carboxylate group connects to the chromanol hydroxyl group of α tocopherol or tocotrienol (e.g. α-tocopherol) and the second carboxylate group connects to a hydroxyl group of a polyoxyethylene chain.

Vitamin E Derivatives—Hydrophilic Portions

The hydrophilic portion of vitamin E derivative containing non-ionic surfactants is typically formed of an oxygen containing moiety that has a hydroxyl group available to form an ester or ether bond with the fatty acid portion (directly or via a short spacer linker), such as an alcohol, or a (poly)alkylene oxide grouping.

A preferred type of hydrophilic portion of vitamin E derivative non-ionic surfactant is a polyoxyethylated vitamin E derivative (sometimes referred to as polyglycolyzed, or PEGylated vitamin E). Polyoxyethylated vitamin E derivatives contain polyoxyethylene groups HO—[CH₂CH₂—O]ₙ— and can be attached directly or indirectly (e.g. through the linkers mentioned above, preferably through a diacid linker such as succinate) to the chromanol hydroxyl group of a tocopherol or tocotrienol.

Polyoxyethylated vitamin E derivatives can be described by the average number of molar equivalents of oxyethylene units, or by the number average molecular weight of the polyoxyethylene portion. The polyoxyethylene portion may contain from 1-180 oxyethylene groups, corresponding to an approximate molecular weight of from about 100 to about 10000 g/mol. For example, the polyoxyethylene portion may contain an average of about 2, 4, 6, 8, 9, 10, 12, 15, 20, 32, 36, 40, 60, 75, 150, or 180, preferably from about 2 to about 75, from about 4 to about 40, more preferably from about 6 to about 32, more preferably from about 8 to about 20 molar equivalents of oxyethylene groups. The polyoxyethylene portion may have a number average molecular weight of about 100, 200, 300, 400, 450, 500, 600, 1000, 1540, 1800, 2000, 3000, 4000, 6000, 8000 g/mol, preferably from about 100 to about 4000, more preferably from about 200 to about 4000, more preferably from about 300 to about 1540, more preferably from about 400 to about 1000 g/mol. In a particularly preferred embodiment, the average molecular weight of the polyoxyethylene chain is 1000 g/mol.

Vitamin E Derivatives—Examples

Examples of vitamin E derivative non-ionic surfactants include: PEG 2-100 (e.g. PEG 4, 6, 8, 9, 10, 12, 15, 20, 32, 36, 40, 60, 75, preferably 10, 12, 15, 20, 32) α-tocopherol succinate (for example PEG-20 d-α-tocopherol succinate, also known as d-α-tocopheryl polyethylene glycol 1000 succinate), PEG 2-100 (e.g. PEG 4, 6, 8, 9, 10, 12, 15, 20, 32, 36, 40, 60, 75 preferably 10, 12, 15, 20, 32) β-tocopheryl succinate, PEG 2-100 (e.g. PEG 4, 6, 8, 9, 10, 12, 15, 20, 32, 36, 40, 60, 75 preferably 10, 12, 15, 20, 32), γ-tocopheryl succinate, PEG 2-100 (e.g. PEG 4, 6, 8, 9, 10, 12, 15, 20, 32, 36, 40, 60, 75 preferably 10, 12, 15, 20, 32) α-tocotrienyl succinate (for example d-α-tocotrienyl polyethylene glycol 1000 succinate), PEG 2-100 (e.g. PEG 4, 6, 8, 9, 10, 12, 15, 20, 32, 36, 40, 60, 75 preferably 10, 12, 15, 20, 32) β-tocotrienyl succinate, PEG 2-100 (e.g. PEG 4, 6, 8, 9, 10, 12, 15, 20, 32, 36, 40, 60, 75 preferably 10, 12, 15, 20, 32), γ-tocopheryl succinate. Any other linker described above, e.g. oxalate, malonate, glutarate, adipate, can be used in place of succinate.

PEG 10-30 α-tocopherol succinates are preferred, in particular d-α-tocopherol polyethylene glycol 1000 succinate (also known as PEG-20 d-α-tocopherol succinate), which is a particularly preferred non-ionic surfactant in the composition of the invention.

Non-ionic surfactants containing one or more vitamin E derivatives such as d-α-tocopherol polyethylene glycol 1000 succinate may additionally contain any of the other vitamin E derivatives, fatty acid esters, fatty alcohol ethers, glycerol derivatives, sorbitan derivatives, or co-polymers listed herein. For example, the additional components may be Solutol HS15; Macrogol cetostearyl ether; Cremaphor EL, Cremaphor RH35, Cremaphor RH40; Labrasol ALF; Labrafac PC; Labrafil M 1944; Labrafil 2125; Gelucire 44/14, Gelucire 50/13; Tween 40; Tween 60; Tween 80; Softisan 378, Poloxamer P124; and mixtures thereof. The mixture of non-ionic surfactants may have a HLB greater than about 5, for example greater than about 10, for example from about 10 to about 20, from about 12 to about 18, or from about 12 to 16.

Fatty Acid Esters

The non-ionic surfactant may comprise fatty acid esters. These are typically derived from a hydrophilic portion containing an alcohol and a hydrophobic portion containing a carboxylic acid, where the linker is an ester bond. Typically, the fatty acid part of the ester forms the hydrophobic portion (e.g. a carboxylic acid with an optionally substituted alkyl chain typically containing 8-30 carbons), with the hydrophilic portion being formed from the alcohol part. The fatty acid ester may have a HLB greater than about 5, for example greater than about 10, for example from about 10 to about 20, from about 12 to about 18, or from about 12 to 16.

Fatty Acid Esters—Hydrophobic Portion

The fatty acid ester typically has a hydrophobic portion comprising a linear or branched 3 to 30 carbon chain. The carbon chain may optionally substituted with one or more hydroxyl, C₁-C₆ alkylene hydroxyl, C₁-C₆ alkoxyl, and/or C₁-C₆ alkyl groups, and may be saturated (no double bonds) or unsaturated (one or more cis or trans double bonds). In some embodiments, the carbon chain can be defined by a “C:D” ratio, where “C” is the total amount of carbon atoms of the fatty acid, and “D” is the number of double bonds in it. Where D>1 it is assumed that the double bonds are separated by one or more methylene bridge(s). Fatty acid esters may have C=3 to 30, and D=0 to 10, for example C=8 to 22, D=0 to 6, more preferably C=8 to 20, D=0 to 4, more preferably C=12 to 18, D=0 to 1.

In addition to those mentioned above, exemplary hydrophobic portions of fatty acid esters contain the following carboxylate moieties (C:D ratios): Caprylate (8:0), Caprate (10:0), Laurate (12:0), Myristate (14:0), Palmitate (16:0), Stearate (18:0), Arachidate (20:0), Myristoleate (14:1), Palmitoleate (16:1), Sapienate (16:1), Oleate (18:1), Elaidate (18:1), Vaccenate (18:1), Linoleate (18:2), Linoelaidate acid (18:2), α-Linolenate (18:3), etc. The carbon chain may optionally substituted with one or more hydroxyl, C₁-C₆ alkylene hydroxyl, C₁-C₆ alkoxyl, and/or C₁-C₆ alkyl groups. Preferred fatty acid esters contain laurate, palmitate, stearate and/or oleate.

The fatty acid ester may be derived from short-chain fatty acids (SOFA), where C=0 to 5. The fatty acid ester may be derived from medium-chain fatty acids (MCFA), where C=6 to 12. The fatty acid ester may be derived from long-chain fatty acids (LCFA), where C=13 to 21. The fatty acid ester may be derived from very long chain fatty acids (VLCFA), where C=22 or greater. Preferred are fatty acid esters are derived from MCFA and/or LCFA, preferably LCFA.

Fatty Acid Esters—Hydrophilic Portion

The hydrophilic portion of fatty acid ester containing non-ionic surfactants is typically formed of an oxygen containing moiety that has a hydroxyl group available to form an ester bond with the fatty acid portion, such as an alcohol, or a (poly)alkylene oxide grouping.

For example, the hydrophilic portion may comprise, consist of, or consist essentially of one or more diols to make diol fatty acid esters. Diols can form one or two linkages (e.g. each could be an ester or an ether linkage) with the hydrophobic portion. An example of a diol in a hydrophilic portion is propylene glycol (propane 1,2-diol) or propane 1,3 diol. Diols can form one or two linkages (e.g. each could be an ester or an ether linkage) with the hydrophobic portion. Exemplary diesters include those made with an alkylene glycol such as ethylene or propylene glycol, for example alkylene, ethylene or propylene glycol dicaprylate, dicaprate, dilaurate, dimyristate, dipalmitate, distearate, etc. (see above). Alternatively, the non-ionic surfactant may be a mixture of a fatty acid monoester and a fatty acid diester.

The hydrophilic portion may comprise, consist of, or consist essentially of polyhydric alcohols (polyols) such as sugar alcohols (to make sugar fatty acid esters or sugar esters), and saccharides (to make saccharide fatty acid esters or saccharide esters), attached to the hydrophobic portion through one or more available hydroxyl groups. Any polyol hydrophilic portion can be combined with any fatty acid or hydrophobic portion listed above.

A preferred type of hydrophilic portion of fatty acid ester non-ionic surfactant is a polyoxyethylated fatty acid ester (sometimes referred to as a polyglycolyzed, or PEGylated fatty acid ester). Polyoxyethylated fatty acid esters contain polyoxyethylene groups HO—[CH₂CH₂O]ₙ— attached to the carboxylate group of the fatty acid hydrophobic portion to form one or two ester bond linkage. The length of the polyoxyethylene group can be varied to fine tune the surfactant properties of the surfactant.

Polyoxyethylated fatty acid esters can be described by the average number of molar equivalents of oxyethylene units, or by the number average molecular weight of the polyoxyethylene portion. The polyoxyethylene portion may contain from 1-180 oxyethylene groups, corresponding to an approximate molecular weight of from about 100 to about 10000 g/mol. For example, the polyoxyethylene portion may contain an average of about 2, 4, 6, 8, 9, 10, 12, 15, 20, 32, 36, 40, 60, 75, 150, or 180, preferably from about 2 to about 75, from about 4 to about 40, more preferably from about 6 to about 32, more preferably from about 8 to about 20 molar equivalents of oxyethylene groups. The polyoxyethylene portion may have a number average molecular weight of about 100, 200, 300, 400, 450, 500, 600, 1000, 1540, 1800, 2000, 3000, 4000, 6000, 8000 g/mol, preferably from about 100 to about 4000, more preferably from about 200 to about 4000, more preferably from about 300 to about 1540, more preferably from about 400 to about 1000 g/mol.

Polyoxyethylated fatty acid esters may contain any chain length of fatty acid and any chain length of polyoxyethylene described above. For example, the fatty acid portion may have C=3 to 30, and D=0 to 10, for example C=8 to 22, D=0 to 6, for example C=8 to 20, D=0 to 4, for example C=12 to 18, D=0 to 1, while the polyoxyethylene portion may contain an average of from about 2 to about 180, for example 2, 4, 6, 8, 9, 10, 12, 15, 20, 32, 36, 40, 60, 75, 150, or 180, for example from about 2 to about 75, for example from about 4 to about 40, for example from about 6 to about 32, for example from about 8 to about 20 molar equivalents of oxyethylene groups.

Fatty Acid Esters—Examples

Examples of non-ionic surfactant may comprise, consist of or consist essentially of: (1) fructose laurate, fructose palmitate, fructose stearate and/or fructose oleate, (2) glucose laurate, glucose palmitate, glucose stearate and/or glucose oleate, (3) sucrose laurate, sucrose palmitate, sucrose stearate and/or sucrose oleate, (4) erythritol laurate, erythritol palmitate, erythritol stearate, and/or (5) erythritol oleate and/or mannitol laurate, mannitol palmitate, mannitol stearate and/or mannitol oleate. In any of (1)-(5) the laurate, palmitate, stearate, and/or oleate may be a mono-, di-, or tri-laurate, palmitate, stearate, and/or oleate, or a mixture of mono-, di-, and tri-esters.

Examples of polyoxyethylated fatty acid esters include: PEG 2-100 (e.g. PEG 4, 6, 8, 9, 10, 12, 15, 20, 32, 36, 40, 60, 75) Caprylate (for example PEG monocaprylates such as Capryol 90 or Capryol PGMC), PEG 2-100 (e.g. PEG 4, 6, 8, 9, 10, 12, 15, 20, 32, 36, 40, 60, 75) Caprate; PEG 2-100 (e.g. PEG 4, 6, 8, 9, 10, 12, 15, 20, 32, 36, 40, 60, 75) Laurate; PEG 2-100 (e.g. PEG 4, 6, 8, 9, 10, 12, 15, 20, 32, 36, 40, 60, 75) Myristate; PEG 2-100 (e.g. PEG 4, 6, 8, 9, 10, 12, 15, 20, 32, 36, 40, 60, 75) Palmitate; PEG 2-100 (e.g. PEG 4, 6, 8, 9, 10, 12, 15, 20, 32, 36, 40, 60, 75) Stearate (for example PEG-esters of 12-hydroxystearate (as found in e.g. Solutol HS15); PEG 2-100 (e.g. PEG 4, 6, 8, 9, 10, 12, 15, 20, 32, 36, 40, 60, 75) Arachidate; PEG 2-100 (e.g. PEG 4, 6, 8, 9, 10, 12, 15, 20, 32, 36, 40, 60, 75) Myristoleate; PEG 2-100 (e.g. PEG 4, 6, 8, 9, 10, 12, 15, 20, 32, 36, 40, 60, 75) Palmitoleate; PEG 2-100 (e.g. PEG 4, 6, 8, 9, 10, 12, 15, 20, 32, 36, 40, 60, 75) Sapienate; and/or PEG 2-100 (e.g. PEG 4, 6, 8, 9, 10, 12, 15, 20, 32, 36, 40, 60, 75) Oleate; and derivatives thereof.

Preferred are PEG-8 to PEG 40 (e.g. PEG 8, 9, 10, 12, 15, 20, 25, 35, 40) acid esters of lauric, palmitic, stearic and/or oleic acid, and derivatives thereof. The carbon chain may optionally substituted with one or more hydroxyl, C₁-C₆ alkylene hydroxyl, C₁-C₆ alkoxyl, and/or C₁-C₆ alkyl groups. Any of the above examples may be a mono-ester. Any of the above examples may be a di-ester. Any of the above examples may be a mixture of mono-esters and di-esters.

Non-ionic surfactants containing one or more fatty acid esters may additionally contain any of the vitamin E derivatives, other fatty acid esters, fatty alcohol ethers, glycerol derivatives, sorbitan derivatives, or co-polymers listed herein. For example, the additional components may be Solutol HS15; Macrogol cetostearyl ether; Cremaphor EL, Cremaphor RH35, Cremaphor RH40; Labrasol ALF; Labrafac PC; Labrafil M 1944; Labrafil 2125; Gelucire 44/14, Gelucire 50/13; Tween 40; Tween 60; Tween 80; Softisan 378, Poloxamer P124; and mixtures thereof. The mixture of non-ionic surfactants may have a HLB greater than about 5, for example greater than about 10, for example from about 10 to about 20, from about 12 to about 18, or from about 12 to 16.

Fatty Alcohol Ethers

The non-ionic surfactant may comprise fatty alcohol ethers. Non-ionic surfactants comprising fatty acid esters are typically composed of a hydrophilic part and a hydrophobic part joined at an ether bond. Fatty alcohol ethers are analogous to fatty acid esters and the properties above apply accordingly.

A preferred type of fatty acid ester to use as a non-ionic surfactant in the composition of the invention is a polyoxyethylated fatty alcohol ether (sometimes referred to as a polyglycolyzed, or PEGylated fatty alcohol ether). Polyoxyethylated fatty acid esters contain polyoxyethylene groups typically attached to a hydroxyl group of the fatty alcohol via an ether linkage. The types of polyethylene groups used with fatty alcohol ethers are the same as those for fatty acid esters.

Examples of polyoxyethylated fatty alcohol ethers include: PEG 2-100 (e.g. PEG 4, 6, 8, 9, 10, 12, 15, 20, 32, 36, 40, 60, 75) Capryl ether; PEG 2-100 (e.g. PEG 4, 6, 8, 9, 10, 12, 15, 20, 32, 36, 40, 60, 75) Capryl ether; PEG 2-100 (e.g. PEG 4, 6, 8, 9, 10, 12, 15, 20, 32, 36, 40, 60, 75) Lauryl ether; PEG 2-100 (e.g. PEG 4, 6, 8, 9, 10, 12, 15, 20, 32, 36, 40, 60, 75) Myristyl ether; PEG 2-100 (e.g. PEG 4, 6, 8, 9, 10, 12, 15, 20, 32, 36, 40, 60, 75) Palmityl ether (often referred to as polyethylene glycol cetyl ether, for example cetomacrogol, e.g. cetomacrogol 1000), PEG 2-100 (e.g. PEG 4, 6, 8, 9, 10, 12, 15, 20, 32, 36, 40, 60, 75) Stearyl ether; PEG 2-100 (e.g. PEG 4, 6, 8, 9, 10, 12, 15, 20, 32, 36, 40, 60, 75) Arachidyl ether; PEG 2-100 (e.g. PEG 4, 6, 8, 9, 10, 12, 15, 20, 32, 36, 40, 60, 75) Myristoleyl ether; PEG 2-100 (e.g. PEG 4, 6, 8, 9, 10, 12, 15, 20, 32, 36, 40, 60, 75) Palmitoleyl ether; PEG 2-100 (e.g. PEG 4, 6, 8, 9, 10, 12, 15, 20, 32, 36, 40, 60, 75) Sapienyl ether; and/or PEG 2-100 (e.g. PEG 4, 6, 8, 9, 10, 12, 15, 20, 32, 36, 40, 60, 75) Oleyl ether; and derivatives thereof. Preferred are PEG-8 to PEG 40 (e.g. PEG 8, 9, 10, 12, 15, 20, 25, 35, 40) fatty alcohol ethers of lauryl, palmityl, stearyl and/or oleyl alcohol, and derivatives thereof. The carbon chain may optionally substituted with one or more hydroxyl, C₁-C₆ alkylene hydroxyl, C₁-C₆ alkoxyl, and/or C₁-C₆ alkyl groups. Any of the above examples may be a mono-ether, a di-ether, or a mixture of mono-ethers and di-ethers.

Non-ionic surfactants containing one or more fatty alcohol ethers may additionally contain any of the vitamin E derivatives, fatty acid esters, other fatty alcohol ethers, glycerol derivatives, sorbitan derivatives, or co-polymers listed herein. For example, the additional components may be Solutol HS15; Macrogol cetostearyl ether; Cremaphor EL, Cremaphor RH35, Cremaphor RH40; Labrasol ALF; Labrafac PC; Labrafil M 1944; Labrafil 2125; Gelucire 44/14, Gelucire 50/13; Tween 40; Tween 60; Tween 80; Softisan 378, Poloxamer P124; and mixtures thereof. The mixture of non-ionic surfactants may have a HLB greater than about 5, for example greater than about 10, for example from about 10 to about 20, from about 12 to about 18, or from about 12 to 16.

Glycerol Derivatives

The non-ionic surfactant may comprise Glycerol derivatives. Non-ionic surfactants comprising Glycerol derivatives are typically composed of a hydrophilic portion containing a glyceryl moiety and joined to a hydrophobic portion via one or more ester or ether bond linkages. Glycerol derivatives may form one, two or three linkages with the hydrophobic portion. The glycerol derivative may have a HLB greater than about 5, for example greater than about 10, for example from about 10 to about 20, from about 12 to about 18, or from about 12 to 16.

Glycerol Derivatives—Hydrophobic Portion

The glyceride typically has a hydrophobic portion comprising a 3 to 30 carbon chain which may be optionally substituted with one or more hydroxyl, C₁-C₆ alkylene hydroxyl, and/or C₁-C₆ alkoxyl groups, and which may be saturated (no double bonds) or unsaturated (one or more cis or trans double bonds). The carbon chain can be defined by a “C:D” ratio, where “C” is the total amount of carbon atoms of the fatty acid, and “D” is the number of double bonds in it. Where D>1 it is assumed that the double bonds are separated by one or more methylene bridge(s). Fatty acid esters may have C=3 to 30, and D=0 to 10, for example C=8 to 22, D=0 to 6, more preferably C=8 to 20, D=0 to 4, more preferably C=12 to 18, D=0 to 1.

In addition to those mentioned above, exemplary hydrophobic portions of glycerides contain the following carboxylate moieties (C:D ratios): Caprylate (8:0, for example as found in Miglyol 812N or Softisan 378), Caprate (10:0, for example as found in Miglyol 812N or Softisan 378), Laurate (12:0), Myristate (14:0), Palmitate (16:0), Stearate (18:0, for example as found in Softisan 378), Arachidate (20:0), Myristoleate (14:1), Palmitoleate (16:1), Sapienate (16:1), Oleate (18:1, e.g. ricinoleate, i.e. 2-hydroxy-9-cis-octadecenoate), Elaidate (18:1), Vaccenate (18:1), Linoleate (18:2), Linoelaidate acid (18:2), α-Linolenate (18:3), etc. The carbon chain may optionally substituted with one or more hydroxyl, C₁-C₆ alkylene hydroxyl, C₁-C₆ alkoxyl, and/or C₁-C₆ alkyl groups. Preferred glycerol esters contain laurate, stearate and/or oleate. The hydrophobic portion of each surfactant molecule may contain one, two, or three carboxylate moieties, for example one, two, or three laurate, one, two, or three stearate and/or one, two, or three oleate moieties.

The fatty acid ester may be derived from short-chain fatty acids (SCFA), where C=0 to 5. The fatty acid ester may be derived from medium-chain fatty acids (MCFA), where C=6 to 12. The fatty acid ester may be derived from long-chain fatty acids (LCFA), where C=13 to 21. The fatty acid ester may be derived from very long chain fatty acids (VLCFA), where C=22 or greater. Preferred are fatty acid esters are derived from MCFA and/or LCFA, preferably LCFA.

Glycerol Derivatives—Hydrophilic Portion

The hydrophilic portion of glyceryl-containing non-ionic surfactant may comprise, consist of, or consist essentially of a glyceryl moiety. In some embodiments, the presence of the glyceryl group alone (which contains three non-ionisable oxygen-containing groups) is sufficient to provide the surfactant with the required hydrophilic properties. In such embodiments, the glyceryl group is attached to the hydrophobic portion directly, for example through one, two, or three ester and/or ether bonds, preferably ester bonds.

In preferred embodiments the hydrophilic portion of the glyceride-containing non-ionic surfactant contains polyethylene glycol. In such polyglycolyzed glycerides, the polyethylene glycol chain is connected to one, two, or three of the hydroxyl groups on the glyceride. The polyoxyethylene glycol forms an ether bond with the glyceryl moiety to form the hydrophilic portion and an ester or ether bond that constitutes a linkage to the hydrophobic portion.

The polyoxyethylene portion may contain a number average of from about 1 to about 180 oxyethylene groups (n=1-180), corresponding to an approximate average molecular weight of from about 50 to about 10000 g/mol. For example, the polyoxyethylene portion may contain a number average of about 2, 4, 6, 8, 9, 10, 12, 15, 20, 32, 36, 40, 60, 75, 150, or 180, preferably from about 2 to about 75, from about 4 to about 40, more preferably from about 6 to about 32, more preferably from about 8 to about 20 molar equivalents of oxyethylene groups. The polyoxyethylene portion may have a number average molecular weight of about 100, 200, 300, 400, 450, 500, 600, 1000, 1540, 1800, 2000, 3000, 4000, 6000, 8000 g/mol, preferably from about 100 to about 4000, more preferably from about 200 to about 4000, more preferably from about 300 to about 1540, more preferably from about 400 to about 1000 g/mol.

Glycerol Derivatives—Examples

Examples of glycerol derivatives include polyoxyethylated glycerides such as PEG 2-100 (e.g. PEG 4, 6, 8, 9, 10, 12, 15, 20, 32, 36, 40, 60, 75) capryl mono-, di- and tri-glycerides, (e.g. PEG-8 capric and caprylic glycerides such as Labrasol ALF), PEG 2-100 (e.g. PEG 4, 6, 8, 9, 10, 12, 15, 20, 32, 36, 40, 60, 75) capryl mono-, di- and tri-glycerides (e.g. PEG-8 capric and caprylic glycerides such as Labrasol ALF); PEG 2-100 (e.g. PEG 4, 6, 8, 9, 10, 12, 15, 20, 32, 36, 40, 60, 75) lauryl mono-, di- and tri-glycerides; PEG 2-100 (e.g. PEG 4, 6, 8, 9, 10, 12, 15, 20, 32, 36, 40, 60, 75) myristyl mono-, di- and tri-glycerides; PEG 2-100 (e.g. PEG 4, 6, 8, 9, 10, 12, 15, 20, 32, 36, 40, 60, 75) palmityl mono-, di- and tri-glycerides; PEG 2-100 (e.g. PEG 4, 6, 8, 9, 10, 12, 15, 20, 32, 36, 40, 60, 75) stearyl mono-, di- and tri-glycerides; PEG 2-100 (e.g. PEG 4, 6, 8, 9, 10, 12, 15, 20, 32, 36, 40, 60, 75) arachidyl mono-, di- and tri-glycerides; PEG 2-100 (e.g. PEG 4, 6, 8, 9, 10, 12, 15, 20, 32, 36, 40, 60, 75) myristoleyl mono-, di- and tri-glycerides; PEG 2-100 (e.g. PEG 4, 6, 8, 9, 10, 12, 15, 20, 32, 36, 40, 60, 75) palmitoleyl mono-, di- and tri-glycerides; PEG 2-100 (e.g. PEG 4, 6, 8, 9, 10, 12, 15, 20, 32, 36, 40, 60, 75) sapienyl mono-, di- and tri-glycerides; and/or PEG 2-100 (e.g. PEG 4, 6, 8, 9, 10, 12, 15, 20, 32, 36, 40, 60, 75) oleyl mono-, di- and tri-glycerides; and derivatives thereof.

Preferred are PEG-8 to PEG 40 (e.g. PEG 8, 9, 10, 12, 15, 20, 25, 35, 40) lauryl mono-, di- and tri-glycerides (e.g. as contained in GELUCIRE 44/14), PEG-8 to PEG 40 (e.g. PEG 8, 9, 10, 12, 15, 20, 25, 35, 40) stearyl mono-, di- and tri-glycerides (e.g. as contained in GELUCIRE 50/13), and PEG-8 to PEG 40 (e.g. PEG 8, 9, 10, 12, 15, 20, 25, 35, 40) oleyl mono-, di- and tri-glycerides. The carbon chain may optionally substituted with one or more hydroxyl, C₁-C₆ alkylene hydroxyl, C₁-C₆ alkoxyl, and/or C₁-C₆ alkyl groups.

The polyoxyethylated glycerides may be monoglycerides. The polyoxyethylated glycerides may be diglycerides. The polyoxyethylated glycerides may be triglycerides. The polyoxyethylated glycerides may be a mixture of monoglycerides and diglycerides. The polyoxyethylated glycerides may be a mixture of diglycerides and triglycerides. Preferably, the polyoxyethylated glycerides are a mixture of monoglycerides, diglycerides and triglycerides. The PEG number in the embodiments can refer to the length of each individual PEG chain, or to the molar equivalence of oxyethylene units compared to the number of moles of glyceride, preferably the molar equivalence of oxyethylene units compared to the number of moles of glyceride.

In one embodiment, the glyceryl-containing non-ionic surfactant contains a polyglycolysed natural oil, for example castor oil, hydrogenated castor oil, vegetable oils such as corn oil, olive oil, peanut oil, palm oil, apricot oil, and almond oil. Preferred are polyoxyethylated castor oil and hydrogenated castor oil. The polyethylene glycol inserts into the glyceride ester bonds in these natural triglycerides.

Examples of polyoxyethylated glycerides prepared from natural oils include PEG 2-100 (e.g. PEG 4, 6, 8, 9, 10, 12, 15, 20, 32, 36, 40, 60, 75) castor oil (e.g. Cremaphor EL), PEG 2-100 (e.g. PEG 4, 6, 8, 9, 10, 12, 15, 20, 32, 36, 40, 60, 75) hydrogenated castor oil (e.g. Cremaphor RH 40); PEG 2-100 (e.g. PEG 4, 6, 8, 9, 10, 12, 15, 20, 32, 36, 40, 60, 75) corn oil (for example PEG-6 corn oil such as Labrafil 2125); PEG 2-100 (e.g. PEG 4, 6, 8, 9, 10, 12, 15, 20, 32, 36, 40, 60, 75) olive oil; PEG 2-100 (e.g. PEG 4, 6, 8, 9, 10, 12, 15, 20, 32, 36, 40, 60, 75) peanut oil; PEG 2-100 (e.g. PEG 4, 6, 8, 9, 10, 12, 15, 20, 32, 36, 40, 60, 75) palm oil; PEG 2-100 (e.g. PEG 4, 6, 8, 9, 10, 12, 15, 20, 32, 36, 40, 60, 75) apricot oil (for example PEG-6 apricot kernel oil such as Labrafil 1944); PEG 2-100 (e.g. PEG 4, 6, 8, 9, 10, 12, 15, 20, 32, 36, 40, 60, 75) almond oil; PEG 2-100 (e.g. PEG 4, 6, 8, 9, 10, 12, 15, 20, 32, 36, 40, 60, 75), and derivatives thereof. Preferred is PEG-6 to PEG 40 (e.g. PEG 8, 9, 10, 12, 15, 20, 25, 35, 40) castor oil (e.g. PEG-35 castor oil such as Cremaphor EL), and PEG-6 to PEG 40 (e.g. PEG 8, 9, 10, 12, 15, 20, 25, 35, 40) hydrogenated castor oil (e.g. PEG40 hydrogenated castor oil such as Cremaphor RH 40). The PEG number in the embodiments can refer to the length of each individual PEG chain, or to the molar equivalence of oxyethylene units compared to the number of moles of glyceride, preferably to the molar equivalence of oxyethylene units compared to the number of moles of glyceride.

Glycerol derivatives as defined above may also be described by Formula (4) shown below:

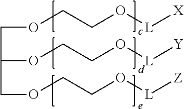

wherein:
L is any linker described herein, for example an ester or an ether linker;
X, Y and Z are each independently any hydrophobic portion described herein, for example a linear or branched saturated or unsaturated 3-30 carbon chain optionally substituted with one or more hydroxyl, C₁-C₆ alkylene hydroxyl, C₁-C₆ alkoxyl, and/or C₁-C₆ alkyl groups, for example a carbon chain having C=4 to 30, and D=0 to 10, for example C=8 to 22, D=0 to 6, more preferably C=8 to 20, D=0 to 4, more preferably C=12 to 18, D=0 to 1;
L-X, L-Y and L-Z may each independently be present or absent, provided that at least one of L-X, L-Y and L-Z is present, for example: L-X and L-Y can be present and L-Z can be absent; L-X and L-Z can be present and L-Y can be absent; L-Y and L-Z can be present and L-X can be absent; or L-X, L-Y and L-Z can all be present;
each of c, d, and e and/or the sum of c+d+e may be from 0-180, for example, the polyoxyethylene portion may contain a number average of about 2, 4, 6, 8, 9, 10, 12, 15, 20, 32, 36, 40, 60, 75, 150, or 180, preferably from about 2 to about 75, from about 4 to about 40, more preferably from about 6 to about 32, more preferably from about 8 to about 20

When c is 0, L-X is directly linked to the glyceryl moiety; c can also be 1-180 as defined above. When d is 0, L-Y is directly linked to the glyceryl moiety; d can also be 1-180 as defined above. When e is 0, L-Z is directly linked to the glyceryl moiety; e can also be 1-180 as defined above.

L-X, L-Y and L-Z can be the same or different. Each of X, Y and Z can independently be a carbon chain having C=4 to 30, and D=0 to 10, for example C=8 to 22, D=0 to 6, more preferably C=8 to 20, D=0 to 4, more preferably C=12 to 18, D=0 to 1. For example, X, Y and Z can be an optionally substituted carbon chain with a C:D of 3:0, 4:0, 5:0, 6:0, 7:0, 8:0, 9:0, 10:0, 11:0, 12:0, 13:0, 14:0, 15:0, 16:0, 17:0, 18:0, 19:0, 20:0, 21:0, 22:0, 23:0, 24:0, 25:0, 26:0, 27:0, 28:0, 29:0, 30:0, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 4:2, 5:2, 6:2, 7:2, 8:2, 9:2, 10:2, 11:2, 12:2, 13:2, 14:2, 15:2, 16:2, 17:2, 18:2, 19:2, 20:2, 21:2, 22:2, 23:2, 24:2, 25:2, 26:2, 27:2, 28:2, 29:2, 30:2, 6:3, 7:3, 8:3, 9:3, 10:3, 11:3, 12:3, 13:3, 14:3, 15:3, 16:3, 17:3, 18:3, 19:3, 20:3, 21:3, 22:3, 23:3, 24:3, 25:3, 26:3, 27:3, 28:3, 29:3, 30:3, 8:4, 9:4, 10:4, 11:4, 12:4, 13:4, 14:4, 15:4, 16:4, 17:4, 18:4, 19:4, 20:4, 21:4, 22:4, 23:4, 24:4, 25:4, 26:4, 27:4, 28:4, 29:4, 30:4, etc. and isomers and derivatives thereof. L-X may be an optionally substituted carbon chain with a C:D of 6:0, 8:0, 10:0, 12:0, 14:0, 16:0, 18:0, or 18:1. L-Y can be an optionally substituted carbon chain with a C:D of 6:0, 8:0, 10:0, 12:0, 14:0, 16:0, 18:0, or 18:1. L-Z may be can be an optionally substituted carbon chain with a C:D of 6:0, 8:0, 10:0, 12:0, 14:0, 16:0, 18:0, or 18:1.

Non-ionic surfactants containing one or more glycerol derivatives may additionally contain any of the vitamin E derivatives, fatty acid esters, fatty alcohol ethers, other glycerol derivatives, sorbitan derivatives, or co-polymers listed herein. For example, the additional components may be Solutol HS15; Macrogol cetostearyl ether; Cremaphor EL, Cremaphor RH35, Cremaphor RH40; Labrasol ALF; Labrafac PC; Labrafil M 1944; Labrafil 2125; Gelucire 44/14, Gelucire 50/13; Tween 40; Tween 60; Tween 80; Softisan 378, Poloxamer P124; and mixtures thereof. The mixture of non-ionic surfactants may have a HLB greater than about 5, for example greater than about 10, for example from about 10 to about 20, from about 12 to about 18, or from about 12 to 16.

Sorbitan Derivatives

The non-ionic surfactant may comprise sorbitan derivatives, such as sorbitan esters. Sorbitan, or 2-(1,2-Dihydroxyethyl)tetrahydrofuran-3,4-diol, is a polyhydric alcohol, and so the hydrophilic portion typically comprises the sorbitan group in these non-ionic surfactant. The hydrophobic group is typically a long carbon chain as defined above, and can be attached by ester or ether linkage. Preferably, the linkage is at the 1-, and/or 2-position on the dihydroxyethyl portion of sorbitan, although the 3- and 4-ring hydroxyl groups are also available for ester or ether linkage. The sorbitan derivative may have a HLB greater than about 5, for example greater than about 10, for example from about 10 to about 20, from about 12 to about 18, or from about 12 to 16

Sorbitan Derivatives—Hydrophobic Portion

Sorbitan esters/ethers typically has a hydrophobic portion comprising a 3 to 30 carbon chain which may be optionally substituted with one or more hydroxyl, C₁-C₆ alkylene hydroxyl, and/or C₁-C₆ alkoxyl groups, and which may be saturated (no double bonds) or unsaturated (one or more cis or trans double bonds). The carbon chain can be defined by a “C:D” ratio, where “C” is the total amount of carbon atoms of the fatty acid, and “D” is the number of double bonds in it. Where D>1 it is assumed that the double bonds are separated by one or more methylene bridge(s). Fatty acid esters may have C=3 to 30, and D=0 to 10, for example C=8 to 22, D=0 to 6, more preferably C=8 to 20, D=0 to 4, more preferably C=12 to 18, D=0 to 1.

In addition to those mentioned above, exemplary hydrophobic portions of sorbitan esters contain the following carboxylate moieties (C:D ratios): Caprylate (8:0), Caprate (10:0), Laurate (12:0), Myristate (14:0), Palmitate (16:0), Stearate (18:0), Arachidate (20:0), Myristoleate (14:1), Palmitoleate (16:1), Sapienate (16:1), Oleate (18:1, e.g. ricinoleate, i.e. 2-hydroxy-9-cis-octadecenoate), Elaidate (18:1), Vaccenate (18:1), Linoleate (18:2), Linoelaidate acid (18:2), α-Linolenate (18:3), etc. The carbon chain may optionally substituted with one or more hydroxyl, C₁-C₈ alkylene hydroxyl, C₁-C₆ alkoxyl, and/or C₁-C₆ alkyl groups. Preferred glycerol esters contain laurate, stearate and/or oleate. The hydrophobic portion of each surfactant molecule may contain one, two, three, or four carboxylate moieties, for example one, two, three or four laurate, one, two, three, or four stearate and/or one, two, three, or four oleate moieties.

Sorbitan Derivatives—Hydrophilic Portion

The hydrophilic portion of sorbitan-containing non-ionic surfactant comprises, consists of, or consists essentially of a sorbitan moiety. In some embodiments, the presence of the sorbitan group alone (which contains four non-ionisable oxygen-containing groups) is sufficient to provide the surfactant with the required hydrophilic properties. In such embodiments, the sorbitan group is attached to the hydrophobic portion directly, for example through one, two, three, or four ester and/or ether bonds, preferably ester bonds).

In preferred embodiments the hydrophilic portion of the sorbitan-containing non-ionic surfactant contains polyethylene glycol. In such polyglycolyzed sorbitans (called polysorbates), the polyethylene glycol chain is connected to one, two, three or four of the hydroxyl groups on the sorbitan The polyoxyethylene glycol typically forms an ether bond with the glyceryl moiety to form the hydrophilic portion and an ester or ether bond that constitutes a linkage to the hydrophobic portion.

The polyoxyethylene portion may contain a number average of from about 1 to about 180 oxyethylene groups (n=1-180), corresponding to an approximate average molecular weight of from about 50 to about 10000 g/mol. For example, the polyoxyethylene portion may contain a number average of about 2, 4, 6, 8, 9, 10, 12, 15, 20, 32, 36, 40, 60, 75, 150, or 180, preferably from about 2 to about 75, from about 4 to about 40, more preferably from about 6 to about 32, more preferably from about 8 to about 20 molar equivalents of oxyethylene groups. The polyoxyethylene portion may have a number average molecular weight of about 100, 200, 300, 400, 450, 500, 600, 1000, 1540, 1800, 2000, 3000, 4000, 6000, 8000 g/mol, preferably from about 100 to about 4000, more preferably from about 200 to about 4000, more preferably from about 300 to about 1540, more preferably from about 400 to about 1000 g/mol.

Sorbitan Derivatives—Examples

Examples of polysorbates include PEG 2-100 (e.g. PEG 4, 6, 8, 9, 10, 12, 15, 20, 32, 36, 40, 60, 75) caprylate, PEG 2-100 (e.g. PEG 4, 6, 8, 9, 10, 12, 15, 20, 32, 36, 40, 60, 75) sorbitan caprate; PEG 2-100 (e.g. PEG 4, 6, 8, 9, 10, 12, 15, 20, 32, 36, 40, 60, 75) sorbitan laurate; PEG 2-100 (e.g. PEG 4, 6, 8, 9, 10, 12, 15, 20, 32, 36, 40, 60, 75) sorbitan myristate; PEG 2-100 (e.g. PEG 4, 6, 8, 9, 10, 12, 15, 20, 32, 36, 40, 60, 75) sorbitan palmitate; PEG 2-100 (e.g. PEG 4, 6, 8, 9, 10, 12, 15, 20, 32, 36, 40, 60, 75) sorbitan stearate; PEG 2-100 (e.g. PEG 4, 6, 8, 9, 10, 12, 15, 20, 32, 36, 40, 60, 75) sorbitan arachidate; PEG 2-100 (e.g. PEG 4, 6, 8, 9, 10, 12, 15, 20, 32, 36, 40, 60, 75) sorbitan myristoleate; PEG 2-100 (e.g. PEG 4, 6, 8, 9, 10, 12, 15, 20, 32, 36, 40, 60, 75) sorbitan palmitoleate; PEG 2-100 (e.g. PEG 4, 6, 8, 9, 10, 12, 15, 20, 32, 36, 40, 60, 75) sorbitan sapienate; and/or PEG 2-100 (e.g. PEG 4, 6, 8, 9, 10, 12, 15, 20, 32, 36, 40, 60, 75) sorbitan oleate; and derivatives thereof.

Preferred are PEG-8 to PEG 40 (e.g. PEG 8, 9, 10, 12, 15, 20, 25, 35, 40) sorbitan laurate (e.g. Tween 20), PEG-8 to PEG 40 (e.g. PEG 8, 9, 10, 12, 15, 20, 25, 35, 40) sorbitan palmitate (e.g. Tween-40), PEG-8 to PEG 40 (e.g. PEG 8, 9, 10, 12, 15, 20, 25, 35, 40) sorbitan stearate (e.g. Tween-60), and PEG-8 to PEG 40 (e.g. PEG 8, 9, 10, 12, 15, 20, 25, 35, 40) sorbitan oleate (e.g. Tween-80). The carbon chain may optionally substituted with one or more hydroxyl, C₁-C₆ alkylene hydroxyl, C₁-C₆ alkoxyl, and/or C₁-C₆ alkyl groups. The PEG number in the embodiments can refer to the length of each individual PEG chain, or to the molar equivalence of oxyethylene units compared to the number of moles of sorbitan, preferably to the molar equivalence of oxyethylene units compared to the number of moles of sorbitan, preferably.

Polysorbates described above may also represented by Formula (5) shown below:

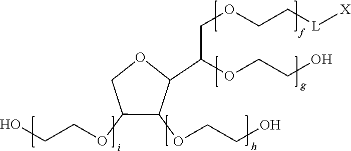

wherein:
L is any linker described herein, for example an ester or an ether linker;
X is any hydrophobic portion described herein, for example a linear or branched saturated or unsaturated 3-30 carbon chain optionally substituted with one or more hydroxyl, C₁-C₆ alkylene hydroxyl, C₁-C₆ alkoxyl, and/or C₁-C₆ alkyl groups, for example a carbon chain having C=4 to 30, and D=0 to 10, for example C=8 to 22, D=0 to 6, more preferably C=8 to 20, D=0 to 4, more preferably C=12 to 18, D=0 to 1;
each of f, g, h, and i, and/or the sum of f+g+h+i may be from 0-40, for example 4, 6, 8, 10, 12, 14, 16, 18, 20, 22, 24, 26, 28, 30, 32, 34, 36, 38, or 40, for example from 4 to 30, or from 6 to 20.

When f is 0, the hydrophobic group is directly linked to the sorbitan moiety f can also be 1-40 as defined above. In the invention, g may be 0-40 as defined above, for example 0. In the invention, h may be 0-40 as defined above, for example 0. In the invention, i may be 0-40 as defined above, for example 0.

Non-ionic surfactants containing one or more sorbitan derivatives may additionally contain any of the vitamin E derivatives, fatty acid esters, fatty alcohol ethers, glycerol derivatives, other sorbitan derivatives, or co-polymers listed herein. For example, the additional components may be Solutol HS15; Macrogol cetostearyl ether; Cremaphor EL, Cremaphor RH35, Cremaphor RH40; Labrasol ALF; Labrafac PC; Labrafil M 1944; Labrafil 2125; Gelucire 44/14, Gelucire 50/13; Tween 40; Tween 60; Tween 80; Softisan 378, Poloxamer P124; and mixtures thereof. The mixture of non-ionic surfactants may have a HLB greater than about 5, for example greater than about 10, for example from about 10 to about 20, from about 12 to about 18, or from about 12 to 16.

Copolymers

The non-ionic surfactant may comprise co-polymers. A preferred class of co-polymer non-ionic surfactant is a POE-POP block co-polymer (referred to as poloxamers). These co-polymers contain a polyoxypropylene hydrophobic portion, and a polyoxyethylene hydrophilic portion, connected by one or more (typically two) ether linkages. Poloxamers can be represented by Formula (6):

HO(C₂H₄O)ₐ(C₃H₆O)_{b}(C₂H₄O)ₐH

Where “a” and “b” represent the number of polyoxyethylene and polyoxypropylene units, respectively. Some commonly available poloxamers are provided in Table 1 below:

TABLE 1
Types of Poloxamer

| Poloxamer | | |
|---|---|---|
| no. | a | b |
| 105 | 11 | 16 |
| 108 | 46 | 16 |
| 122 | 5 | 21 |
| 123 | 7 | 21 |
| 124 | 11 | 21 |
| 181 | 3 | 30 |
| 182 | 8 | 30 |
| 183 | 10 | 30 |
| 184 | 13 | 30 |
| 185 | 19 | 30 |
| 188 | 75 | 30 |
| 212 | 8 | 35 |
| 215 | 24 | 35 |
| 217 | 52 | 35 |
| 231 | 16 | 39 |
| 234 | 22 | 39 |
| 235 | 27 | 39 |
| 237 | 62 | 39 |
| 238 | 97 | 39 |
| 282 | 10 | 47 |
| 284 | 21 | 47 |
| 288 | 122 | 47 |
| 331 | 7 | 54 |
| 333 | 20 | 54 |
| 334 | 31 | 54 |
| 335 | 38 | 54 |
| 338 | 128 | 54 |
| 401 | 6 | 67 |
| 402 | 13 | 67 |
| 403 | 21 | 67 |
| 407 | 98 | 67 |

The sorbitan derivative may have a HLB greater than about 5, for example greater than about 10, for example from about 10 to about 20, from about 12 to about 18, or from about 12 to 16.

Non-ionic surfactants containing one or more co-polymers may additionally contain any of the vitamin E derivatives, fatty acid esters, fatty alcohol ethers, glycerol derivatives, sorbitan derivatives, or other co-polymers listed herein. For example, the additional components may be Solutol HS15; Macrogol cetostearyl ether; Cremaphor EL, Cremaphor RH35, Cremaphor RH40; Labrasol ALF; Labrafac PC; Labrafil M 1944; Labrafil 2125; Gelucire 44/14, Gelucire 50/13; Tween 40; Tween 60; Tween 80; Softisan 378, Poloxamer P124; and mixtures thereof. The mixture of non-ionic surfactants may have a HLB greater than about 5, for example greater than about 10, for example from about 10 to about 20, from about 12 to about 18, or from about 12 to 16.

In any embodiment herein, the composition containing non-ionic surfactants having linked hydrophilic and hydrophobic portion may additionally the free, unlinked hydrophilic and hydrophobic portions of that non-ionic surfactant or any other listed herein.

Co-Solvents

The compositions of the invention may include one or more co-solvents. Co-solvents include alcohols, such as propylene glycol, polyethylene glycol, ethanol, glycerin, 2-(2-Ethoxyethoxy)ethanol (TRANSCUTOL) and mixtures thereof. Preferred co-solvents include PEG 300, PEG 1000 and 2-(2-Ethoxyethoxy)ethanol. The concentration of co-solvent maybe between 1% to 25% w/w of the total weight of the composition, for example about 20% w/w of the total weight. In some embodiments, the addition of one or more co-solvents may reduce the viscosity of the composition, which may be desirable during processing (e.g. when using an automated capsule filling machine).

One co-solvent that may be used with the invention is polyethylene glycol. The polyoxyethylene portion may contain a number average of from about 1 to about 180 oxyethylene groups (n=1-180), corresponding to an approximate average molecular weight of from about 50 to about 10000 g/mol. For example, the polyoxyethylene portion may contain a number average of about 2, 4, 6, 8, 9, 10, 12, 15, 20, 32, 36, 40, 60, 75, 150, or 180, preferably from about 2 to about 75, from about 4 to about 40, more preferably from about 6 to about 32, more preferably from about 8 to about 20 molar equivalents of oxyethylene groups. The polyoxyethylene portion may have a number average molecular weight of about 100, 200, 300, 400, 450, 500, 600, 1000, 1540, 1800, 2000, 3000, 4000, 6000, 8000 g/mol, preferably from about 100 to about 4000, more preferably from about 200 to about 4000, more preferably from about 300 to about 1540, more preferably from about 400 to about 1000 g/mol.

Anti-Oxidants

The compositions of the invention may comprise one or more antioxidants in order to minimize or eliminate the oxidative degradation of the formulation, e.g. the oxidative degradation of the compound of Formula (1), the non-ionic surfactant, and/or any additional excipients.

Thus, the pharmaceutical compositions of the invention may comprise an antioxidant such as: ascorbic acid or a derivative thereof; citric acid or a derivative thereof; sodium metabisulfate, sodium thiosulfate; cysteine; tryptophan; methionine; butylated hydroxytoluene; propyl, octyl, dodecyl esters of gallic acid, or a combination thereof. For example, the antioxidant may be ascorbic acid, cysteine or sodium metabisulphate. The antioxidant may be present in an amount of up to about 0.1% by weight of the composition.

Treatment with Compositions of the Invention

Pharmaceutical compositions as defined herein may be for use in therapy.

The pharmaceutical compositions of the invention contain an inhibitor of Aurora kinase and FLT3 activity. Thus, the present invention provides a method of inhibiting Aurora kinase activity and/or FLT3 in a human or animal subject in need of such inhibition, the method comprising administering to said subject an effective amount of the pharmaceutical composition of the invention. The composition may be for use in such methods, i.e. for use in the treatment of disease or condition associated with Aurora kinase activity (and/or FLT3 activity).

The invention also provides a composition as defined herein for use in method of inhibiting Aurora kinase activity and/or FLT3 in a human or animal subject in need of such inhibition, the method comprising administering to said subject an effective amount of the pharmaceutical composition of the invention. The Aurora kinase may be Aurora kinase A, B or C.

The present invention provides a method of treating a proliferative disorder in a human or animal subject, the method comprising administering to said subject a therapeutically acceptable amount of the pharmaceutical composition of the invention. The composition may be for use in such methods, i.e. for use in the treatment of a proliferative disorder. For example, the pharmaceutical composition of the invention is for use in the treatment of cancer, in particular the treatment of acute myeloid leukemia (AML).

### Aspects of the Invention

1. A pharmaceutical composition, comprising:
   a) a compound according to Formula (1):

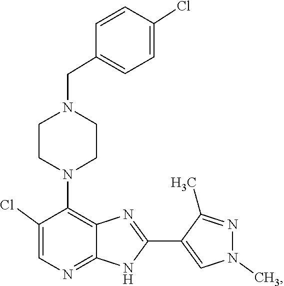 [SMILES: Cc1nn(C)cc1-c1nc2c(N3CCN(Cc4ccc(Cl)cc4)CC3)c(Cl)cnc2[nH]1]

or a pharmaceutically acceptable salt, and/or solvate thereof; and
b) a non-ionic surfactant.
2. The pharmaceutical composition according to aspect 1 wherein the non-ionic surfactant is represented by the Formula (2):

Hydrophilic portion-linker(s)-hydrophobic portion
wherein the hydrophilic portion contains one or more moieties comprising non-ionisable oxygen-containing groups such as alcohols and/or ethers;
the hydrophobic portion contains one or more linear or branched, saturated or unsaturated 3-30 carbon chains optionally substituted with one or more hydroxyl, C₁-C₆ alkylene hydroxyl, C₁-C₆ alkoxyl, and/or C₁-C₆ alkyl groups; and
the linker or linkers contain one or more ester and/or ether bonds, and may be a separate chemical moiety or may derive from the connection of existing groups in the hydrophilic and a hydrophobic portions.
3. The pharmaceutical composition according to aspect 1 or 2, wherein the non-ionic surfactant has a HLB value of greater than 5, for example greater than 10, for example from about 10 to about 18, about 10 to about 16, about 12 to about 18, or about 12 to about 16.
4. The pharmaceutical composition according to aspect 2 or 3, wherein the non-ionisable oxygen-containing groups include monohydric alcohols, dihydric alcohols, trihydric alcohols, polyhydric alcohols, and/or polymers of alkylene glycols for example polyethylene oxide.
5. The pharmaceutical composition according to aspect 1, 2, 3, or 4 wherein the non-ionic surfactant has a hydrophilic portion containing polyoxyethylene groups having the Formula (3):

—O—[CH₂CH₂—O]ₙ—
wherein n refers to the number average of oxyethylene groups, and is from about 2 to about 180.
6. The pharmaceutical composition according to aspect 5, wherein the average molecular weight of polyoxyethylene groups is from about 50 to about 10000 g/mol.
7. The pharmaceutical composition according to aspect 5 or 6, wherein n is from about 2 to about 75, from about 4 to about 60, from about 6 to about 50, from about 8 to about 40, from about 10 to about 40, from about 15 to about 30, or about 20.
8. The pharmaceutical composition according to aspect 7 wherein n is about 4 to about 60.
9. The pharmaceutical composition according to aspect 8 wherein n is from about 6 to about 50.
10. The pharmaceutical composition according to aspect 9 wherein n is from about 8 to about 40.
11. The pharmaceutical composition according to aspect 10 wherein n is from about 10 to about 40.
12. The pharmaceutical composition according to aspect 11, wherein n is from about 15 to about 30.
13. The pharmaceutical composition according to aspect 4, wherein the non-ionic surfactant has a hydrophilic portion contain monohydric alcohols, dihydric alcohols, trihydric alcohols, or polyhydric alcohols and one, two, three, or four polyoxyethylene chains as described in aspects 5 to 12.
14. The pharmaceutical composition according to any preceding aspect, wherein the hydrophobic portion is derived from a vitamin E, a polypropylene glycol, a carboxylic acid, or an alcohol.
15. The pharmaceutical composition according to aspect 14, wherein the hydrophobic portion is derived from a vitamin E, for example an α tocopherol such as d-α tocopherol.
16. The pharmaceutical composition according to aspect 14, wherein the hydrophobic portion is derived from a polypropylene glycol.
17. The pharmaceutical composition according to aspect 14, wherein the hydrophobic portion is derived from a carboxylic acid.
18. The pharmaceutical composition according to aspect 14, wherein the hydrophobic portion is derived from an alcohol.
19. The pharmaceutical composition according to aspect 17 or 18, wherein the hydrophobic portion comprises a C3 to C30 carbon chain wherein:
   a) C=4 to 30, and D=0 to 10,
   b) C=8 to 22, and D=0 O to 6,
   c) C=8 to 20 and D=0 to 4, or
   d) C=12 to 18, D=0 to 1.
20. The pharmaceutical composition according to aspect 17 or 18, wherein the hydrophobic portion comprises a C3 to C30 carbon chain wherein C:D is 3:0, 4:0, 5:0, 6:0, 7:0, 8:0, 9:0, 10:0, 11:0, 12:0, 13:0, 14:0, 15:0, 16:0, 17:0, 18:0, 19:0, 20:0, 21:0, 22:0, 23:0, 24:0, 25:0, 26:0, 27:0, 28:0, 29:0, 30:0, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 4:2, 5:2, 6:2, 7:2, 8:2, 9:2, 10:2, 11:2, 12:2, 13:2, 14:2, 15:2, 16:2, 17:2, 18:2, 19:2, 20:2, 21:2, 22:2, 23:2, 24:2, 25:2, 26:2, 27:2, 28:2, 29:2, 30:2, 6:3, 7:3, 8:3, 9:3, 10:3, 11:3, 12:3, 13:3, 14:3, 15:3, 16:3, 17:3, 18:3, 19:3, 20:3, 21:3, 22:3, 23:3, 24:3, 25:3, 26:3, 27:3, 28:3, 29:3, 30:3, 8:4, 9:4, 10:4, 11:4, 12:4, 13:4, 14:4, 15:4, 16:4, 17:4, 18:4, 19:4, 20:4, 21:4, 22:4, 23:4, 24:4, 25:4, 26:4, 27:4, 28:4, 29:4, 30:4.
21. The pharmaceutical composition according to aspect 20, wherein the hydrophobic portion comprises Caprylate (8:0), Caprate (10:0), Laurate (12:0), Myristate (14:0), Palmitate (16:0), Stearate (18:0), Arachidate (20:0), Myristoleate (14:1), Palmitoleate (16:1), Sapienate (16:1), Oleate (18:1, e.g. ricinoleate, i.e. 2-hydroxy-9-cis-octadecenoate), Elaidate (18:1), Vaccenate (18:1), Linoleate (18:2), Linoelaidate acid (18:2), α-Linolenate (18:3).
22. The pharmaceutical composition according to aspect 19, 20, or 21, wherein the hydrophobic portion comprises one or more carbon chains having a C:D of 12:0, 16:0, 18:0, or 18:1.
23. The pharmaceutical composition according to any of aspects 2-19 wherein the linker comprises at least one chemical bond, for example an ester bond or an ether bond.
24. The pharmaceutical composition according to any of aspects 2-19 wherein the linker consists of at least one chemical bond, for example an ester bond or an ether bond.
25. The pharmaceutical composition according to any of aspects 2-19 wherein the linker comprises a C1-C10 diol such as ethane diol, propane diol, butane diol, pentane diol, or hexane diol.
26. The pharmaceutical composition according to any of aspects 2-19 wherein the linker comprises a C1-C10 diacid, such as ethanedioic acid, propanedioic acid, butanedioic acid, pentanedioic acid, hexanedioic acid, preferably butanedioic acid.
27. The pharmaceutical composition according to any preceding aspect, wherein the non-ionic surfactant is selected from: vitamin E derivatives, fatty acid esters, fatty alcohol ethers, glycerol derivatives, sorbitan derivatives, block co-polymers, and combinations thereof.
28. The pharmaceutical composition according to aspect 27, wherein the non-ionic surfactant is a vitamin E derivative.
29. The pharmaceutical composition of aspect 28, wherein the vitamin E derivative is a polyalkoxylated vitamin E derivative, for example a polyalkoxylated ester derivative.
30. The pharmaceutical composition of aspect 29, wherein the vitamin E derivative is a polyethoxylated succinate ester.
31. The pharmaceutical composition of aspect 29 or 30, wherein the average number of oxyethylene units is from about 2 to about 75, from about 4 to about 60, from about 6 to about 50, from about 8 to about 40, from about 10 to about 40, from about 15 to about 30, or about 20.
32. The pharmaceutical composition of any preceding aspect, wherein the vitamin E derivative is α tocopherol polyethylene glycol 1000 succinate.
33. The pharmaceutical composition of any preceding aspect, wherein the vitamin E derivative is d-α tocopherol polyethylene glycol 1000 succinate.
34. The pharmaceutical composition of any preceding aspect, wherein the vitamin E derivative is I-α tocopherol polyethylene glycol 1000 succinate.
35. The pharmaceutical composition of any preceding aspect, wherein the vitamin E derivative is dl-α tocopherol polyethylene glycol 1000 succinate.
36. The pharmaceutical composition according to aspect 27, wherein the non-ionic surfactant is a fatty acid ester.
37. The pharmaceutical composition according to aspect 36, wherein the fatty acid ester contains a hydrophobic portion as defined in aspect 19 to 22.
38. The pharmaceutical composition according to aspect 36 or 37, wherein the fatty acid ester contains a hydrophilic portion as defined in any one of aspects 4 to 12.
39. The pharmaceutical composition according to any of aspects 36 to 38, wherein the fatty acid ester is a mono-ester, a di-ester, or a combination of mono-esters and diesters.
40. The pharmaceutical composition according to aspect 27, wherein the non-ionic surfactant is a fatty alcohol ether.
41. The pharmaceutical composition according to aspect 27, wherein the non-ionic surfactant is a glycerol derivative.
42. The pharmaceutical composition according to aspect 41, wherein the glycerol derivative contains a hydrophobic portion as defined in aspect 19 to 22.
43. The pharmaceutical composition according to aspect 41 or 42, wherein the glycerol derivative is polyoxyethylated as defined in any one of aspects 5 to 12.
44. The pharmaceutical composition according to any of aspects 41 to 43, wherein the glycerol derivative is defined according to Formula (4).
45. The pharmaceutical composition according to any of aspects 41 to 44, wherein the glycerol derivative is a mono-glyceride, a di-glyceride, a triglyceride, or a combination thereof.
46. The pharmaceutical composition according to aspect 27, wherein the non-ionic surfactant is a sorbitan derivative.
47. The pharmaceutical composition according to aspect 47, wherein the sorbitan derivative contains a hydrophobic portion as defined in aspect 19 to 22.
48. The pharmaceutical composition according to aspect 46 or 47, wherein the sorbitan derivative is polyoxyethylated as defined in any one of aspects 5 to 12.
49. The pharmaceutical composition according to any of aspects 47 to 48, wherein the sorbitan derivative is defined according to Formula (5).
50. The pharmaceutical composition according to aspect 27, wherein the non-ionic surfactant is a block co-polymer.
51. The pharmaceutical composition according to aspect 50, wherein the block co-polymer is a poloxamer as defined according to Formula (6).
52. The pharmaceutical composition according to aspect 51, wherein the poloxamer number is 105 108 122 123 124 181 182 183 184 185 188 212 215 217 231 234 235 237 238 282 284 288 331 333 334 335 338 401 402 403, or 407.
53. The pharmaceutical composition according to aspect 27, wherein the non-ionic surfactant has a hydrophilic region as defined in aspect 7, 11, or 12, a hydrophobic region as defined in aspect 15, 16 or 22, and a linker as defined in aspect 23 or 26.
54. The pharmaceutical composition according to any preceding aspect, wherein the non-ionic surfactant is selected from: d-α tocopherol polyethylene glycol 1000 succinate; Solutol HS15 (PEG-esters of 12-hydroxystearate); Macrogol cetostearyl ether; Cremaphor EL (PEG-35 castor oil), Cremaphor RH35 (PEG-35 Hydrogenated castor oil), Cremaphor RH40 (PEG-40 Hydrogenated castor oil); Labrasol ALF (PEG-8 Caprylic/capric triglceride); Labrafac PC (PEG dicaprylate/dicaprate); Labrafil M 1944 (PEG-6 apricot kernel oil); Labrafil 2125 (PEG-6 corn oil); Capryol 90 (PEG monocaprylate); Capryol PCMG (PEG monocaprylate); Gelucire 44/14 (PEG-32 laurate with mono-, di-, and tri-glycerides); Gelucire 50/13 (PEG-32 stearate with mono-, di-, and tri-glycerides); Tween 40 (PEG-20 sorbitan monopalmitate); Tween 60 (PEG-20 sorbitan monostearate); Tween 80 (PEG-20 sorbitan monooleate); Softisan 378 (Coconut oil triglycerides, e.g. capric, caprylic, stearic, myristic); Poloxamer P124; and mixtures thereof.
55. The pharmaceutical composition according to any preceding aspect, wherein the non-ionic surfactant is selected from: d-α tocopherol polyethylene glycol 1000 succinate; Solutol HS15; Cremaphor EL, Cremaphor RH40; Labrasol ALF; Gelucire 44/14; Gelucire 50/13; Poloxamer P124; and mixtures thereof.
56. The pharmaceutical composition of any preceding aspect, wherein the composition comprises from about 5% to about 95% of the non-ionic surfactant, for example comprises
   a) from about 20% to about 90% by weight;
   b) from about 40% to about 90% by weight;
   c) from about 60% to about 90% by weight; or
   d) from about 80 to about 90% by weight
   of the non-ionic surfactant.
57. The pharmaceutical composition of any preceding aspect, wherein the composition is a mixture, a liquid-liquid dispersion, a solid-liquid dispersion (e.g. a suspension), a solid-solid dispersion, a semi solid matrix, or a solution.
58. The pharmaceutical composition of aspect 57, wherein the composition is a semi-solid matrix.
59. The pharmaceutical composition according to any preceding aspect, wherein the composition comprises from about 1 mg to about 2 g of the compound of Formula (1).
60. The pharmaceutical composition according to aspect 59, wherein the composition comprises:
   a) from about 1 mg to about 50 mg;
   b) from about 5 mg to about 45 mg;
   c) from about 10 mg to about 35 mg;
   d) from about 10 mg to about 30 mg;
   e) from about 15 mg to about 25 mg; or
   f) about 20 mg
   of the compound of Formula (1).
61. The pharmaceutical composition according to aspect 59, wherein the composition comprises:
   a) from about 50 mg to about 150 mg;
   b) from about 60 mg to about 140 mg;
   c) from about 70 mg to about 130 mg;
   d) from about 80 mg to about 120 mg;
   e) from about 90 mg to about 110 mg; or
   f) about 100 mg
   of the compound of Formula (1).
62. The pharmaceutical composition of any preceding aspect, wherein the ratio of the weight of non-ionic surfactant to the weight of the compound of Formula (1) is from about 1:1 to about 25:1, for example:
   a) from about 2:1 to about 25:1;
   b) from about 5:1 to about 20:1;
   c) from about 7:1 to about 20:1;
   d) from about 1:1 to about 10:1;
   e) from about 1:1 to about 8:1; or
   f) from about 2:1 to about 7:1.
63. The pharmaceutical composition of any preceding aspect, wherein the compound of Formula (1) is milled.
64. The pharmaceutical composition of any preceding aspect, wherein the compound of Formula (1) is non-milled.
65. The pharmaceutical composition of any preceding aspect, wherein the composition is an oral dosage form.
66. The pharmaceutical composition of any preceding aspect, wherein the composition is a tablet, capsule, or pill.
67. The pharmaceutical composition of any preceding aspect, wherein the composition is a capsule, for example a softgel capsule or a hardgel capsule.
68. The pharmaceutical composition of aspect 67, wherein the composition is a softgel capsule.
69. The pharmaceutical composition of aspect 67, wherein the composition is a hardgel capsule.
70. The pharmaceutical composition of any preceding aspect, wherein the compound of Formula (1) is a free base.
71. The pharmaceutical composition of any preceding aspect, wherein the compound of Formula (1) is a fumarate salt.
72. The pharmaceutical composition of any preceding aspect, wherein the composition comprises one or more additional pharmaceutically acceptable carriers, diluents and excipients.
73. The pharmaceutical composition of aspect 72, wherein the composition comprises a co-solvent such as propylene glycol, polyethylene glycol, ethanol, glycerin, 2-(2-Ethoxyethoxy)ethanol) and mixtures thereof.
74. The pharmaceutical composition of aspect 73, wherein the composition comprises polyethylene glycol, for example PEG 300.
75. The pharmaceutical composition of any preceding aspect, wherein the composition comprises an antioxidant, such as: ascorbic acid or a derivative thereof; citric acid or a derivative thereof; sodium metabisulfate, sodium thiosulfate; cysteine; tryptophan; methionine; butylated hydroxytoluene; propyl, octyl, dodecyl esters of gallic acid, or a combination thereof.
76. A pharmaceutical composition as defined in any preceding aspect for use in therapy.
77. A pharmaceutical composition as defined in any preceding aspect for use in the treatment of disease or condition associated with Aurora kinase activity (and/or FLT3 activity).
78. A pharmaceutical composition as defined in any preceding aspect for use in a method of inhibiting Aurora kinase activity and/or FLT3 in a human or animal subject in need of such inhibition, the method comprising administering to said subject an effective amount of a pharmaceutical composition as defined in any of aspects 1-74.
79. A method of treating a proliferative disorder in a human or animal subject, the method comprising administering to said subject a therapeutically acceptable amount of a pharmaceutical composition as defined in any of aspects 1-74, for example wherein the proliferative disorder is cancer, such as acute myeloid leukemia (AML).
80. A method of preparing a composition as defined in any of aspects 1-75.
81. The method of aspect 80, comprising by mixing, dissolving, dispersing, suspending, spray drying, melting, tabletting, compacting, a compound of Formula (1) with a non-ionic surfactant.
82. The use of a compound of Formula (1) in the manufacture of a medicament comprising a composition as defined in any of aspects 1-75.

### EXAMPLES

### Example 1—UHPLC Development

Initially, quantities of the compound of Formula (1) were analysed using the Ultra High Performance Liquid Chromatography (UHPLC) method set out below:

TABLE 2
UHPLC conditions-acid method

| Item | Value |
|---|---|
| Instrument | Waters Acquity UPLC |
| Column | Acquity UPLC CSH C-18 2.1 × 100 mm |
| Column temperature | 60° C. |
| Mobile phase A | Water |
| Mobile phase B | Acetonitrile |
| Mobile phase C | 5% formic acid |
| Injection | 10 μL |
| Detection wavelength | PDA 190-450 nm (254 nm) |

TABLE 3
UHPLC Gradient program-acid method

| | Flow rate | | | |
|---|---|---|---|---|
| Time | (mL/min) | % A | % B | % C |
| Initial | 1 | 96 | 2 | 2 |
| 0.2 | 1 | 96 | 2 | 2 |
| 2.5 | 1 | 0 | 98 | 2 |
| 3.2 | 1 | 0 | 98 | 2 |
| 3.5 | 1 | 96 | 2 | 2 |

However, analysis of the dissolution samples revealed that poor chromatography was resulting in erroneously high values being determined for the dissolved amount of the compound of Formula (1). The reason for this was determined to be insufficient ion suppression of the compound of Formula (1) during chromatography, resulting in the compound of Formula (1) eluting as two different peaks dependent on the sample composition (acid or FaSSIF). To avoid this the method was developed resulting in the LC eluent being adjusted to a higher pH and the samples re-analysed. The conditions for the modified LC method are given in Tables 4 and 5. The modification to the eluent pH gave an improved, quantitative, chromatographic method. The improved method was used for all experiments carried out.

TABLE 4
UHPLC conditions-modified basic method

| Item | Value |
|---|---|
| Instrument | Waters Acquity UPLC |
| Column | Acquity UPLC CSH C-18 2.1 × 100 mm |
| Column temperature | 60° C. |
| Mobile phase A | Water |
| Mobile phase B | Acetonitrile |
| Mobile phase C | 5% ammonium hydroxide |
| Injection | 10 μL |
| Detection wavelength | PDA 190-450 nm (254 nm) |

TABLE 5
UHPLC Gradient program-modified basic method

| | Flow rate | | | |
|---|---|---|---|---|
| Time | (mL/min) | % A | % B | % C |
| Initial | 1 | 96 | 2 | 2 |
| 0.2 | 1 | 96 | 2 | 2 |
| 2.5 | 1 | 0 | 98 | 2 |
| 3.2 | 1 | 0 | 98 | 2 |
| 3.5 | 1 | 96 | 2 | 2 |

### Example 2—Solubility of the Compound of Formula (1) in Non-Ionic Surfactants

Approximately 3 mg of the compound of Formula (1) (monofumarate) was weighed out into clean HLPC vials. Various non-ionic surfactants were added in 200 or 300 μL portions up to 1 ml in total. After each addition, the sample was with vortexed (2×30 seconds) sonicated for 6 minutes, and once 1 ml total had been added the sample was kept in an oven at 45° C. for 40 minutes before being cooled to RT to aid dissolution. Observations are provided below:

TABLE 6
Solubility in non-ionic surfactants

| Non-ionic surfactant | Non-ionic | Solubility |
|---|---|---|
| type | surfactant | (mg/ml) |
| Sorbitan derivative | Tween 80 | <3.7 |
| Fatty acid ester | Labrafac PC | <2.1 |
| Fatty acid | Gelucire 44/14 | <3.5 |
| ester/glycerol | | |
| derivative | | |
| Fatty acid | Gelucire 50/13 | <2.7 |
| ester/glycerol | | |
| derivative | | |
| Glycerol derivative | Labrasol ALF | <3.5 |
| Fatty acid ester | Capryol 90 | <4.6 |
| Fatty acid ester | Capryol PC MG | <3.6 |
| Glycerol derivative | Miglyol 812N | <3.2 |
| Co-polymer | Kollisolv P124 | <4.5 |
| Glycerol derivative | Cremophor EL | <4.9 |
| Glycerol derivative | Cremophor RH40 | <4.4 |
| Vitamin E derivative | Vitamin E TPGS | <3.2 |
| Glycerol derivative | Softisan 378 | <4.3 |
| Glycerol derivative | Labrafil 1944 | <5.0 |
| Glycerol derivative | Labrafil 2125 | <5.5 |

Those excipients that have a melting point above ambient temperature were heated to above their melting point (e.g. 50° C.-70° C.) and observations were made in the liquid phase.

### Example 3—Excipient Solubilising Ability

Promising non-ionic surfactants were further tested. Each was prepared as a 20% w/v aqueous solution. A portion (100 μL) of a concentrated solution of the compound of Formula (1) (free base) in DMSO was added to 900 μL of each excipient solution to give a final volume of 1 mL and the mixture was observed for signs of precipitation. If precipitation was observed the concentration of the compound in DMSO was reduced and the experiment repeated until no precipitation was observed in a least one excipient.

The excipients tested were (HLB): Cremophor EL (12-14); Cremophor RH 40 (14-16); Labrasol ALF; Solutol HS15 (˜16); Poloxamer P124 (12-18); TPGS (˜13); and Gelucire 44/14 (˜11).

Initially, the concentrated DMSO solution was prepared at 30 mg/mL to give a starting concentration of 3 mg/mL of the compound of Formula (1) in the aqueous vehicle, and the concentration was reduced until a semi-solid excipient was identified which maintained a clear solution of the compound. No excipients solubilised the compound at concentrations >1.5 mg/mL in this experiment, but TPGS unexpectedly performed best—solubilising the compound at a concentration of 1.5 mg/mL in the aqueous vehicle (e.g. 100 μL portion of a 15 mg/mL solution of the compound in DMSO was added to each excipient solution to achieve the final concentration of 1.5 mg/mL). In TPGS the solution remained clear for up to 5 hours. Interestingly, although no other excipient gave a clear solution at 1.5 mg/mL, other excipients were able to provide stable suspensions: for example both Poloxamer P124 and Gelucire 44/14 produced suspensions which remained stable for at least 10 days.

Poloxamer P124 and TPGS were selected for further development on account of these promising results.

### Example 4—Preparation of Formulations

A suspension of the compound of Formula (1) (fumarate) in Poloxamer P124 was prepared at a nominal concentration of 150 mg/g. From this, size 00 capsules were filled so that each capsule contained 100 mg of the compound of Formula (1) as a free base equivalent (125 mg fumarate salt). Similarly, a second set of size 00 capsules were filled with a suspension of the compound of Formula (1) fumarate prepared in TPGS at the same nominal concentration. The composition of each formulation is given in Tables 7 and 8 below.

Manufacture of Poloxamer P124 based capsules were performed at ambient conditions. Poloxamer P124 was weighed into a suitable container. The compound of Formula (1) fumarate was added gradually with both components being continuously mixed using a magnetic stirrer and magnetic flea to produce a visually uniform distribution of drug substance ideally with no observable lumps or agglomerates. The mixture was blended continuously for at least 3 hours. The blend was then filled manually into size 00 gelatin-PEG capsule shells to the target capsule fill weight (Table 7). Filling of the capsules was performed using a pasture pipette.

The compound of Formula (1) fumarate TPGS capsules were manufactured on a small scale using standard blending and manual capsule filling processes. Vitamin E polyethylene glycol succinate is melted with a product temperature not less than 60° C. The compound of Formula (1) fumarate salt is then added gradually with both components being continuously mixed together using a hotplate stirrer and magnetic stirrer to produce a visually uniform distribution of the drug substance ideally with no observable lumps or agglomerates. The mixture was blended continuously for at least 2 hours. The blend is then maintained in the molten state and filled manually into size 00 gelatin-PEG capsule shells to the target capsule fill weight (Table 8).

TABLE 7
Poloxamer 124 based 100 mg formulation composition

| | Quantity per | Composition for |
|---|---|---|
| | 100 mg | 100 mg Capsules |
| Component | Capsule (mg)* | (% w/w) |
| Compound of | 125.00* | 15.00 |
| Formula (1) | | |
| fumarate* | | |
| Poloxamer P124 | 708 | 85.00 |
| Size 00 PGH opaque | 1 capsule | — |
| yellow 67 | | |
| Quali-G PEG | | |
| Total per capsule | 833 | 100.00% |

*100 mg free base

TABLE 8
TPGS based 100 mg formulation composition

| | Quantity | Composition for |
|---|---|---|
| | per 100 mg | 100 mg Capsules |
| Component | Capsule (mg)* | (% w/w) |
| Compound of Formula | 125.00* | 15.00 |
| (1) fumarate* | | |
| Vitamin E polyethylene | 708 | 85.00 |
| glycol succinate | | |
| (TPGS) | | |
| Size 00 PGH opague | 1 capsule | — |
| yellow 67 | | |
| Quali-G PEG | | |
| Total per capsule | 833 mg | 100.00% |

*100 mg free base

### Example 5—Dissolution Performance—in Acid with pH Shift to FaSSIF

A pH shift dissolution test was carried out on each of the formulations described in Example 4 using USP Type 2 dissolution apparatus as detailed in Table 9. At time zero a capsule was placed into each dissolution vessel containing 250 mL of pH 1.2 buffer heated to 37° C. and stirred by a paddle rotating at 75 rpm. A 5 mL sample was removed after 15, 30, 45 and 60 minutes. For each 5 mL sample removed, 5 mL of media was added to keep the volume at 250 mL. After the 60 minutes time point, 250 mL of double concentration FaSSIF (Fasted State Simulated Intestinal Fluid) was added. A 5 mL sample was then removed after 75, 90, 105, 120, 150 and 180 minutes. For this second part of the experiment, the media was not replaced as each sample was taken.

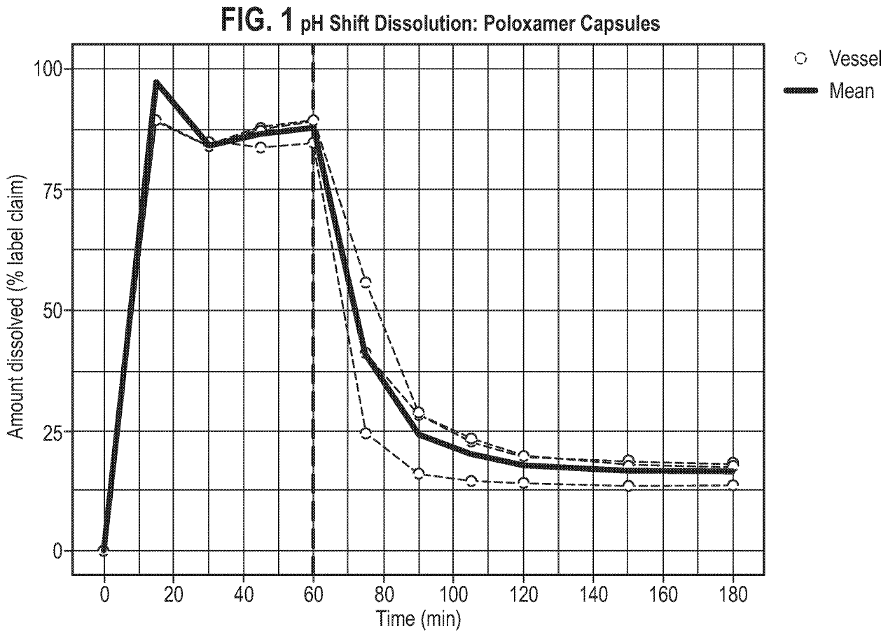

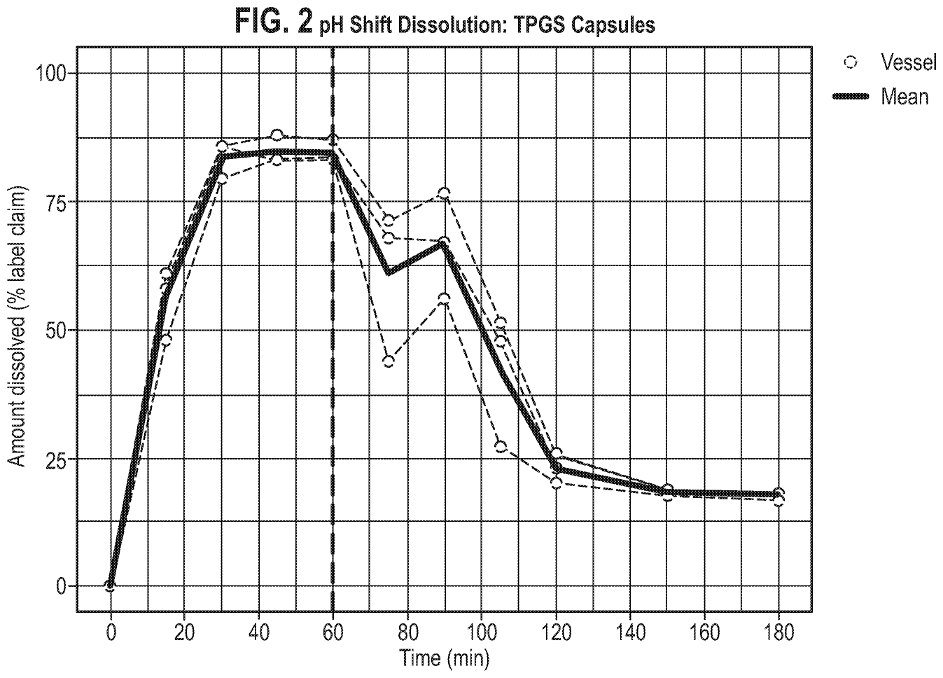

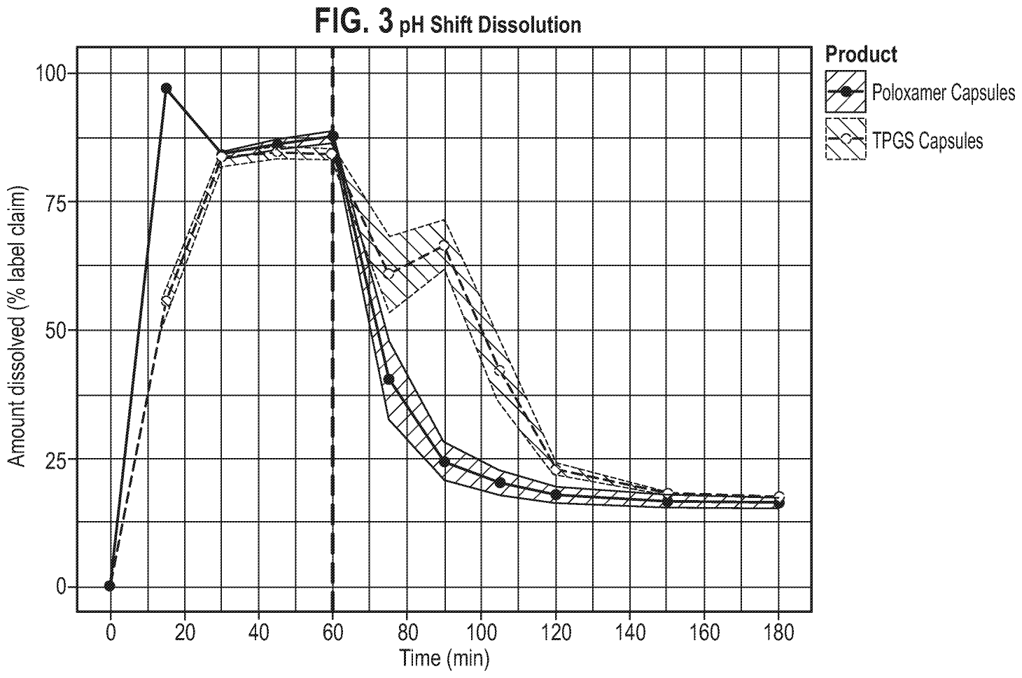

Each sample was filtered through a 0.2 μm PTFE filter then diluted with an equivalent volume of methanol to ensure dissolved compound of Formula (1) remained in solution. The samples were analysed by UPLC using the modified basic method detailed in Example 1 above. Three capsules of each formulation were assessed as part of the pH shift dissolution study, the results are given in the dissolution plots shown in FIG. 1, FIG. 2 and FIG. 3.

TABLE 9
pH shift dissolution conditions

| Dissolution | | |
|---|---|---|
| parameter | Value | |
| Apparatus: | USP 2 | |
| Stir Speed | 75 | rpm |
| Media inc pH: | 0.1M HCl (pH 1.2), | |
| | 2 × FaSSIF (pH 6.5) | |
| Starting Volume: | 250 | mL |
| Final Volume | 500 | mL |
| Temp: | 37° C. | |
| Sample Vol: | 5 mL (replaced for acid | |
| | phase only) | |
| Sinkers: | spiral | |
| Sample times | 15, 30, 45, 60 (pH shift), 75, | |
| | 90, 105, 120, 150 & 180 | |
| | minutes | |
| pH Shift @ | 60 | min |

Results

The poloxamer P124 based formulation containing the compound of Formula (1) fumarate dissolved rapidly in acid. Rapid precipitation was observed on pH change to final compound concentration of ˜13-20 μg/mL, however about 40% (25%-56%) of the compound was still in solution 15 mins post-shift.

The TPGS based formulation containing the compound of Formula (1) fumarate dissolved quite rapidly in acid to ˜85% of label claim. On pH shift precipitation was not immediate (visual observation) and concentration remained supersaturated throughout the experiment with ˜18% remaining in solution 2 hours after pH Shift (final concentration ˜20 μg/mL).

### Example 6—Dissolution Performance—FaSSIF pH 6.5

Dissolution in FaSSIF was also carried out in USP Type 2 dissolution apparatus as detailed in Table 10. One capsule of each formulation containing the compound of Formula (1) fumarate was placed in a dissolution vessel containing 500 mL of FaSSIF pH 6.5 heated to 37° C. and stirred by a paddle rotating at 75 rpm. A 5 mL sample was removed after 0, 15, 30, 45, 60, and after infinity spin (90 min), the media was not replaced as each sample was taken. An infinity spin involves the paddle speed being increased to 250 rpm after the 60 minute samples were taken until 90 minutes. This ensures full dispersion of a formulation and is standard practice in early development when investigating the dissolution behaviour of new drug products.

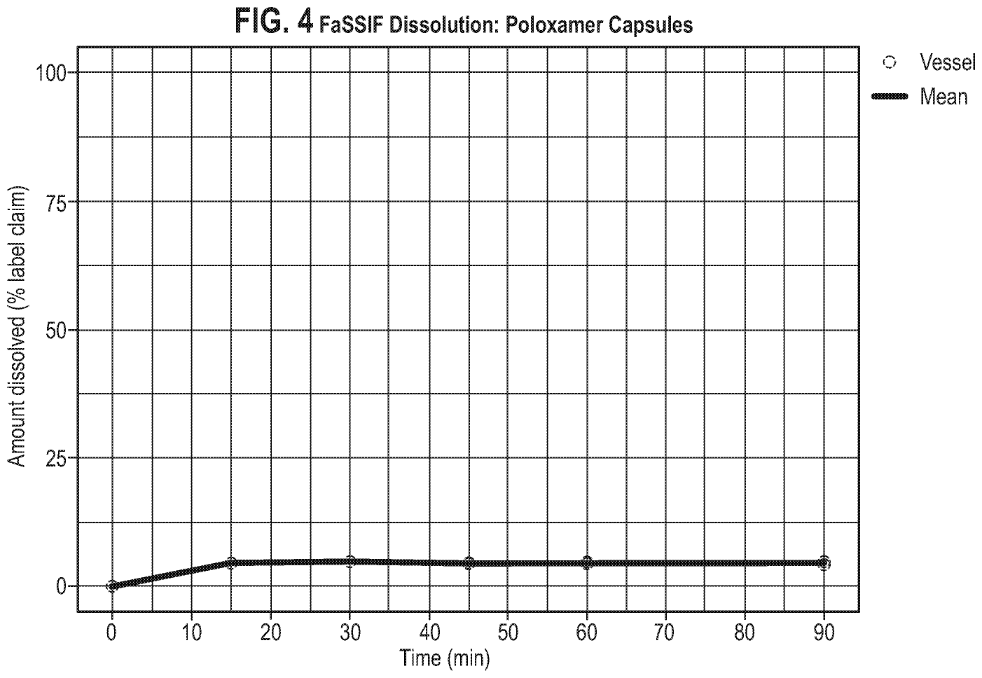

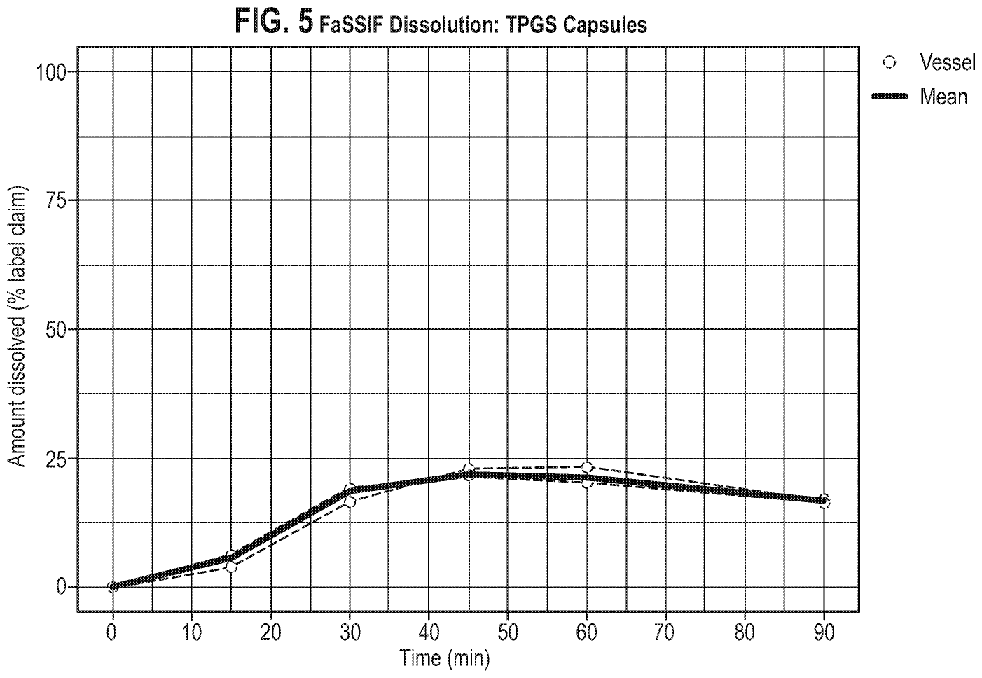

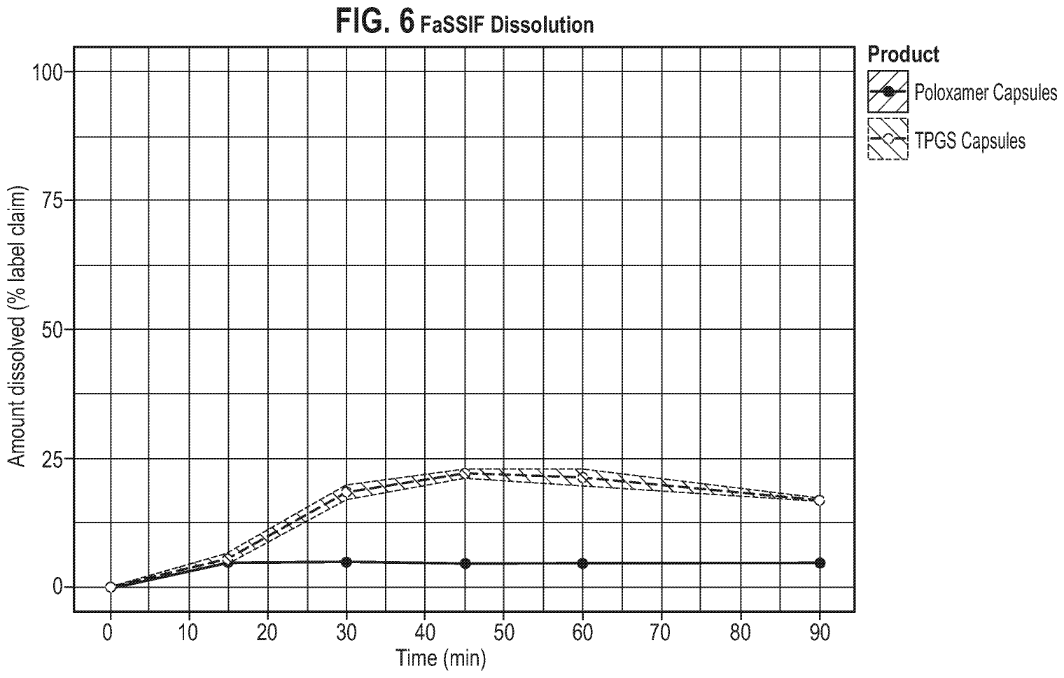

Each sample was filtered through a 0.45 μm PTFE filter then diluted with an equivalent volume of methanol to ensure dissolved compound remained in solution. The samples were analysed by UPLC using the modified basic method described in Example 1. Dissolution was assessed in triplicate for each formulation, the results are given in the dissolution plots shown in FIG. 4, FIG. 5 and FIG. 6.

TABLE 10
FaSSIF dissolution conditions

| Dissolution | | |
|---|---|---|
| parameter | Value | |
| Apparatus: | USP 2 | |
| Stir Speed | 75 | rpm |
| Media inc pH: | FaSSIF, pH 6.5 | |
| Volume: | 500 | mL |
| Temp: | 37° C. | |
| Sample Vol: | 5 mL (no replacement) | |
| Sinkers: | spiral | |
| Sample times | 15, 30, 45, 60 & 90 minutes | |
| Infinity spin after: | 60 | min |

Results

The results are shown in FIGS. 4, 5 and 6. both formulations provided dissolution in FaSSIF, although the TPGS formulation unexpectedly provided a greater concentration of the compound in solution.

The poloxamer P124 based formulation dissolved rapidly in FaSSIF however dissolution plateaued at ˜5% of dose (compound concentration ˜9 μg/mL). The TPGS based formulation dissolved rapidly in FaSSIF with up to ˜22% dissolved at the peak after 45 minutes (compound concentration ˜43 μg/mL). Visually, both formulations were fully dispersed at 60 minutes.

### Example 7—Hard Capsule Stability Test

Stability studies were carried out using fill preparations containing compound of Formula (1) fumarate in TPGS at 15% w/w or 5% w/w (free base content). The fill suspensions were manufactured by dispersing the compound into TPGS previously melted at a temperature of 60-70° C. and stirring for at least two hours at this temperature. The composition of these batches is provided in Table 11. These suspensions were maintained at 60-70° C. and filled into different hard capsule shells as detailed in Table 12. The capsules were filled by hand into empty capsule shells held within a Torpac Profill capsule maker, aided with the use of a semi-automatic Eppendorf pipette which allows multiple dispensing operations from one refill of the pipette. In these trials capsule banding was carried out using a gelatin banding solution for all capsule types, with the exception of LiCap capsules, which are intended for use with liquid fill preparations without the need for banding.

TABLE 11
Composition of 15% w/w and 5% w/w Capsule Fill Suspensions

| Capsule Fill | | Percentage | Batch |
|---|---|---|---|
| Suspension | Material | Formula (% w/w) | Quantity (g) |
| 15% | 6-chloro-7-(4-(4- | 18.815 | 37.63 |
| | chlorobenzyl)piperazin-1-yl)-2- | | |
| | (1,3-dimethyl-1H-pyrazol-4-yl)-3H-imidazo[4,5- | | |
| | b]pyridine fumarate | | |
| | TPGS | 81.185 | 162.37 |
| | Total | 100 | 200.00 |
| 5% | 6-chloro-7-(4-(4- | 6.272 | 12.54 |
| | chlorobenzyl)piperazin-1-yl)-2- | | |
| | (1,3-dimethyl-1H-pyrazol-4-yl)-3H-imidazo[4,5- | | |
| | b]pyridine fumarate | | |
| | TPGS | 93.278 | 187.46 |
| | Total | 100 | 200.00 |

TABLE 12
Filling Trials with 15% w/w and 5% w/w Capsule Fill Suspension

| | Amount of 6-chloro-7-(4-(4- | Target | | |
|---|---|---|---|---|
| | chlorobenzyl)piperazin-1-yl)-2-(1,3- | fill | | |
| Capsule fill | dimethyl-1H-pyrazol-4-yl)-3H- | weight | | |
| Suspension | imidazo[4,5-b]pyridine per capsule (mg) | (mg) | Capsule Shell | Banding |
| 15% | 20 | 135 | Swedish Orange HPMC | Gelatin |
| | 50 | 334 | Swedish Orange Gelatin | Gelatin |
| | 50 | 334 | Transparent HPMC | — |
| | | | LiCaps | |
| | 20 | 135 | Swedish Orange Gelatin | Gelatin |
| 5% | 20 | 400 | Swedish Orange Gelatin | Gelatin |

Capsules from each of filling sub-batch were placed on an accelerated stability protocol for four weeks with testing at initial, one, two and four weeks at 25° C./60% RH and 40° C./75% RH as standard test conditions with 30° C./65% RH and 5° C. storage as contingency. There were no significant changes in the potency and reported related substances observed in the capsules over the time of the study and compared to a control capsule fill solution. There was no indication of any incompatibility of the filled suspension with any of the capsule shells tested. The appearance and feel of some batches encapsulated by gelatin capsule shells and at higher temperature or time points, indicated the possibility of some of the capsule content leaking through the shell material. From these results the use of the HPMC capsule shell format was progressed.

### Example 8—Preparation of 5% w/w, 20 mq Formulation and 10% w/w, 50 mg Formulation

Capsules were prepared according to in Example 7 with the following modifications. The viscosity of the 15% w/w fill suspension was quite high and contributed to variability in dispensing the target fill weight, so a capsule fill suspension of 10% w/w was used. A wider bore visco tip pipette tip was used, and the pipette tip was warmed before use to prevent the fill suspension beginning to solidify around the sides of the tip during the filling operation. In addition, the speed of rotation of the Silverson mixer used for mixing was increased to assist the preparation of a more uniform suspension in the suspension bulk. In order to prevent foaming and the incorporation of too much air in the bulk mix, a Silverson deaerator head was employed. The formulation is displayed in Tables 13 and 14 below.

TABLE 13
20 mg/5% w/w capsule

| Capsule Shells Used: | Size 0, Swedish Orange HPMC | |
|---|---|---|
| Target Fill Weight: | 400 mg | |
| Fill Suspension: | 5% 6-chloro-7-(4-(4-chlorobenzyl)piperazin-1-yl)-2- | |
| | (1,3-dimethyl-1H-pyrazol-4-yl)-3H-imidazo[4,5- | |
| | b]pyridine per capsule | |
| | Percentage | |
| | Formula (% w/w) | Batch Quantity (g) |
| 6-chloro-7-(4-(4- | 6.272 | 15.68 |
| chlorobenzyl)piperazin-1-yl)-2-(1,3- | | |
| dimethyl-1H-pyrazol-4-yl)-3H-imidazo[4,5- | | |
| b]pyridine fumarate | | |
| TPGS | 93.728 | 234.32 |
| Total | 100 | 250 |

TABLE 14
50 mg/10% w/w capsule

| Capsule Shells Used: | Size 0, Swedish Orange HPMC | |
|---|---|---|
| Target Fill Weight: | 500 mg | |
| Fill Suspension: | 10% 6-chloro-7-(4-(4-chlorobenzyl)piperazin-1-yl)-2- | |
| | (1,3-dimethyl-1H-pyrazol-4-yl)-3H-imidazo[4,5- | |
| | b]pyridine per capsule | |
| | Percentage | |
| | Formula (% w/w) | Batch Quantity (g) |
| 6-chloro-7-(4-(4- | 12.543 | 31.36 |
| chlorobenzyl)piperazin-1-yl)-2-(1,3- | | |
| dimethyl-1H-pyrazol-4-yl)-3H-imidazo[4,5- | | |
| b]pyridine fumarate | | |
| TPGS | 87.457 | 218.64 |
| Total | 100 | 250 |

The production of these batches confirmed that the modifications to the process (use of deaerator heard and modifications to filling pipette tips) improved to improve content uniformity for the 20 mg and 50 mg capsules.

1. A pharmaceutical composition, comprising:
a) a compound according to Formula (1): 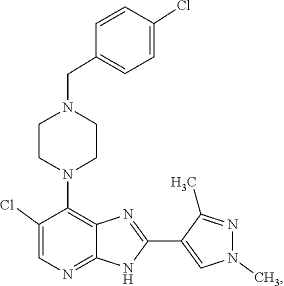 [SMILES: Cc1nn(C)cc1-c1nc2c(N3CCN(Cc4ccc(Cl)cc4)CC3)c(Cl)cnc2[nH]1]
or a pharmaceutically acceptable salt, and/or solvate thereof; and
b) a non-ionic surfactant.

2. The pharmaceutical composition according to claim 1, wherein the non-ionic surfactant is represented by the Formula (2):
hydrophilic portion-linker(s)-hydrophobic portion
wherein the hydrophilic portion contains one or more moieties comprising non-ionisable oxygen-containing groups such as alcohols and/or ethers;
the hydrophobic portion contains one or more linear or branched, saturated or unsaturated 3-30 carbon chains optionally substituted with one or more hydroxyl, C₁-C₆ alkylene hydroxyl, C₁-C₆ alkoxyl, and/or C₁-C₆ alkyl groups; and
the linker or linkers contain one or more ester and/or ether bonds, and may be a separate chemical moiety or may derive from the connection of existing reactive groups in the hydrophilic and a hydrophobic portions.

3. The pharmaceutical composition according to any preceding claim, wherein the non-ionic surfactant is selected from: vitamin E derivatives, fatty acid esters, fatty alcohol ethers, glycerol derivatives, sorbitan derivatives, block co-polymers, and combinations thereof.

4. The pharmaceutical composition according to any of claims 1 to 3, wherein the non-ionic surfactant has a hydrophilic portion containing polyoxyethylene groups having the Formula (3):
—O—[CH₂CH₂—O]ₙ—
wherein n refers to the number average of oxyethylene groups, and is from about 2 to about 180.

5. The pharmaceutical composition according to claim 4, wherein n is from about 2 to about 75, from about 4 to about 60, from about 6 to about 50, from about 8 to about 40, from about 10 to about 40, from about 15 to about 30, or about 20.

6. The pharmaceutical composition according to any preceding claim, wherein the hydrophobic portion is derived from a vitamin E, a polypropylene glycol, a carboxylic acid, or an alcohol.

7. The pharmaceutical composition according to claim 6, wherein the hydrophobic portion comprises a vitamin E, for example α, β, γ, δ tocopherol, or α, β, γ, δ tocotrienol, preferably α tocopherol.

8. The pharmaceutical composition according to claim 6, wherein the hydrophobic portion comprises a C3 to C30 carbon chain having a C:D ratio of 8:0, 10:0, 12:0, 14:0, 16:0, 18:0, 20:0, 14:1, 16:1, 18:1, 18:2, 18:2, 18:3, or more specifically wherein C:D is 12:0, 16:0, 18:0, or 18:1.

9. The pharmaceutical composition according to any of claims 2-8 wherein the linker consists of at least one chemical bond, for example an ester bond or an ether bond, or wherein the linker comprises a C1-C10 diacid, such as butanedioic acid.

10. The pharmaceutical composition according to any preceding claim, wherein the non-ionic surfactant is a vitamin E derivative.

11. The pharmaceutical composition of claim 10, wherein the vitamin E derivative is a polyalkoxylated vitamin E derivative, for example a polyalkoxylated ester derivative, such as a polyethoxylated succinate ester.

12. The pharmaceutical composition of any preceding claim, wherein the vitamin E derivative is α tocopherol polyethylene glycol 1000 succinate, preferably d-α tocopherol polyethylene glycol 1000 succinate.

13. The pharmaceutical composition according to any of claims 1 to 9, wherein the non-ionic surfactant is a fatty acid ester.

14. The pharmaceutical composition according to claim 13, wherein the fatty acid ester contains: a hydrophilic portion comprising an alcohol, or as defined in claim 4 or 5; and/or a hydrophobic portion comprising a carbon chain as defined in claim 8.

15. The pharmaceutical composition according to any of claims 13 to 14, wherein the fatty acid ester is a mono-ester, a di-ester, or a combination of mono-esters and diesters.

16. The pharmaceutical composition according to any of claims 1 to 9, wherein the non-ionic surfactant is a glycerol derivative.

17. The pharmaceutical composition according to claim 16, wherein the glycerol derivative contains: a hydrophilic portion comprising glycerol and optionally polyoxyethylene groups as defined in claim 4 or 5; and/or a hydrophobic portion comprising a carbon chain as defined in claim 8.

18. The pharmaceutical composition according to claim 16 or 17, wherein the glycerol derivative is a mono-glyceride, a di-glyceride, a triglyceride, or a combination thereof.

19. The pharmaceutical composition according to any of claims 1 to 9, wherein the non-ionic surfactant is a sorbitan derivative.

20. The pharmaceutical composition according to claim 19, wherein the sorbitan derivative contains: a hydrophilic portion as defined in claim 4 or 5; and/or a hydrophobic portion comprising a carbon chain as defined in claim 8.

21. The pharmaceutical composition according to any of claim 1 to 7, or 9, wherein the non-ionic surfactant is a block co-polymer, for example wherein the poloxamer number is 105 108 122 123 124 181 182 183 184 185 188 212 215 217 231 234 235 237 238 282 284 288 331 333 334 335 338 401 402 403, or 407, preferably 108.

22. The pharmaceutical composition according to any preceding claim, wherein the non-ionic surfactant has a hydrophilic region as defined in claim 4 or 5, a hydrophobic region as defined in claim 6, 7 or 8, and a linker as defined in claim 9.

23. The pharmaceutical composition according to any preceding claim, wherein the non-ionic surfactant is selected from: d-α tocopherol polyethylene glycol 1000 succinate; Solutol HS15; Macrogol cetostearyl ether; Cremaphor EL, Cremaphor RH35, Cremaphor RH40; Labrasol ALF; Labrafac PC; Labrafil M 1944; Labrafil 2125; Gelucire 44/14, Gelucire 50/13; Tween 40; Tween 60; Tween 80; Softisan 378, Poloxamer P124; and mixtures thereof.

24. The pharmaceutical composition according to any preceding claim, wherein the non-ionic surfactant is selected from: d-α tocopherol polyethylene glycol 1000 succinate; Solutol HS15; Cremaphor EL, Cremaphor RH40; Labrasol ALF; Gelucire 44/14; Gelucire 50/13; Poloxamer P124; and mixtures thereof.

25. The pharmaceutical composition according to any preceding claim, wherein the non-ionic surfactant has a HLB value of greater than 5, for example greater than 10, for example from about 10 to about 18, about 10 to about 16, about 12 to about 18, or about 12 to about 16.

26. The pharmaceutical composition of any preceding claim, wherein the composition comprises from about 5% to about 95% of the non-ionic surfactant, for example comprises
a) from about 20% to about 90% by weight;
b) from about 40% to about 90% by weight;
c) from about 60% to about 90% by weight;
d) from about 80 to about 90% by weight
of the non-ionic surfactant.

27. The pharmaceutical composition of any preceding claim, wherein the composition is a mixture, a liquid-liquid dispersion, a solid-liquid dispersion (e.g. a suspension), a solid-solid dispersion, a semi solid matrix, or a solution, for example wherein the composition is a semi-solid matrix.

28. The pharmaceutical composition according to any preceding claim, wherein the composition comprises from about 1 mg to about 2 g of the compound of Formula (1), for example wherein the composition comprises:
a) from about 1 mg to about 50 mg;
b) from about 5 mg to about 45 mg;
c) from about 10 mg to about 35 mg;
d) from about 10 mg to about 30 mg;
e) from about 15 mg to about 25 mg;
f) about 20 mg;
g) from about 50 mg to about 150 mg;
h) from about 60 mg to about 140 mg;
i) from about 70 mg to about 130 mg;
j) from about 80 mg to about 120 mg;
k) from about 90 mg to about 110 mg; or
I) about 100 mg
of the compound of Formula (1).

29. The pharmaceutical composition of any preceding claim, wherein the ratio of the weight of non-ionic surfactant to the weight of the compound of Formula (1) is from about 1:1 to about 25:1, for example:
a) from about 2:1 to about 25:1;
b) from about 5:1 to about 20:1;
c) from about 7:1 to about 20:1;
d) from about 1:1 to about 10:1;
e) from about 1:1 to about 8:1; or
f) from about 2:1 to about 7:1.

30. The pharmaceutical composition of any preceding claim, wherein the composition is in an oral dosage form, for example a tablet, capsule, or pill, preferably a hardgel capsule.

31. The pharmaceutical composition of any preceding claim, wherein the compound of Formula (1) is a free base or a fumarate salt.

32. The pharmaceutical composition of any preceding claim, wherein the composition comprises one or more additional pharmaceutically acceptable carriers, diluents and excipients.

33. The pharmaceutical composition of claim 32, wherein the composition comprises a co-solvent such as propylene glycol, polyethylene glycol, ethanol, glycerin, 2-(2-Ethoxyethoxy)ethanol) and mixtures thereof.

34. The pharmaceutical composition of claim 32 or 33, wherein the composition comprises an antioxidant such as ascorbic acid, cysteine or sodium metabisulphate.

35. A pharmaceutical composition as defined in any preceding claim for use in therapy.

36. A pharmaceutical composition as defined in any of claims 1 to 34 for use in the treatment of disease or condition associated with Aurora kinase activity (and/or FLT3 activity).

37. A method of treating a proliferative disorder in a human or animal subject, the method comprising administering to said subject a therapeutically acceptable amount of the pharmaceutical composition as defined in any of claims 1 to 34, for example wherein the proliferative disorder is cancer, such as acute myeloid leukemia (AML).

38. A method of preparing a composition as defined in any of claims 1 to 34, for example by mixing, dissolving, dispersing, suspending, spray drying, melting, tabletting, compacting, a compound of Formula (1) with a non-ionic surfactant.

39. The use of a compound of Formula (1) in the manufacture of a medicament comprising a composition as defined in any of claims 1 to 34.
